SEQUENCE LISTING (103 sequences)

SEQ ID NO: 1 - protein, 373 aa, Homo sapiens
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 2 - protein, 373 aa, Cricetulus griseus
MATSASSHLNKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNARRLTGFHETSNINDFSAGVANRSASIRIPRTVGQEKKGYFEDRRPSANCDPFAVTEAIVRTCLLNETGDEPFQYKN

SEQ ID NO: 3 - protein, 373 aa, Mus musculus
MATSASSHLNKGIKQMYMSLPQGEKVQAMYIWVDGTGEGLRCKTRTLDCEPKCVEELPEWNFDGSSTFQSEGSNSDMYLHPVAMFRDPFRKDPNKLVLCEVFKYNRKPAETNLRHICKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWIARFILHRVCEDFGVIATFDPKPIPGNWNGAGCHTNFSTKAMREENGLKCIEEAIDKLSKRHQYHIRAYDPKGGLDNARRLTGFHETSNINDFSAGVANRGASIRIPRTVGQEKKGYFEDRRPSANCDPYAVTEAIVRTCLLNETGDEPFQYKN

SEQ ID NO: 4 - protein, 373 aa, Rattus norvegicus
MATSASSHLNKGIKQMYMNLPQGEKIQLMYIWVDGTGEGLRCKTRTLDCDPKCVEELPEWNFDGSSTFQSEGSNSDMYLHPVAMFRDPFRRDPNKLVFCEVFKYNRKPAETNLRHSCKRIMDMVSSQHPWFGMEQEYTLMGTDGHPFGWPSNGFPGPQGPYYCGVGADKAYGRDIVEAHYRACLYAGIKITGTNAEVMPAQWEFQIGPCEGIRMGDHLWVARFILHRVCEDFGVIATFDPKPIPGNWNGAGCHTNFSTKAMREENGLRCIEEAIDKLSKRHQYHIRAYDPKGGLDNARRLTGFHETSNINDFSAGVANRSASIRIPRIVGQEKKGYFEDRRPSANCDPYAVTEAIVRTCLLNETGDEPFQYKN

SEQ ID NO: 5 - DNA, 8201 bp, synthetic construct
  misc_feature 1..8201: note = Biscistronic mAb GS
  source 1..8201: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgccaccatggacatgagggtccctgctcagctcctggggctcctgctgctctggctctcaggtgccagatgtgacattgtaatgacacagaccccactctccctgcccgtcacccctggagagccggcctccatctcctgcaggtctagtaagagcctcctgcatagtaatggaatcacctatttgtactggtacctgcagaagccagggcagtctccacagctcctgatctatcagatgtctaatctggtgtccggggtccctgacaggttcagtggcagtggatcaggcacagattttacactgaaaatcagcagagtggaggctgaggatgttggggtttattactgcgcccaaaacctagagctgccctacactttcggcggagggaccaaggtggagatcaaacgtaccgtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaggatatcgtctagacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggagtttgggctgagctgggttttcctcgttgctctttttagaggtgtccagtgtcaggtgcagctggtgcagtctggggctgaggtgaagaagcctgggtcctccgtgaaggtctcctgcaaggcctctggatacgccttctcctactcttggatcaactgggtgcgacaggcccctggacaagggctcgagtggatgggacggatcttccctggcgatggtgacacagactacaacggcaagttcaagggcagagtcaccatcaccgccgacaagtccacgagcacagcctacatggaactgagcagcctgagatctgaggacacggccgtctattactgtgcacgaaacgtgttcgatggctattggctggtgtactggggccaggggaccctggtcaccgtcagcagcgctagcaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatcgagtctagagggcccgtttaaacccgctgatcagcctcgactgtgccttctaggcggccgcttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcggccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 588 bp, synthetic construct
  misc_feature 1..588: note = CMV Promoter
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 7 - DNA, 729 bp, synthetic construct
  misc_feature 1..729: note = Light chain of antibody GA101 (LC 1-729)
  source 1..729: mol_type = other DNA
atggacatgagggtccctgctcagctcctggggctcctgctgctctggctctcaggtgccagatgtgacattgtaatgacacagaccccactctccctgcccgtcacccctggagagccggcctccatctcctgcaggtctagtaagagcctcctgcatagtaatggaatcacctatttgtactggtacctgcagaagccagggcagtctccacagctcctgatctatcagatgtctaatctggtgtccggggtccctgacaggttcagtggcagtggatcaggcacagattttacactgaaaatcagcagagtggaggctgaggatgttggggtttattactgcgcccaaaacctagagctgccctacactttcggcggagggaccaaggtggagatcaaacgtaccgtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtag

SEQ ID NO: 8 - DNA, 20 bp, synthetic construct
  misc_feature 1..20: note = Wild type Internal Ribosome Entry Site
  source 1..20: mol_type = other DNA
atgataatatggccacaacc

SEQ ID NO: 9 - DNA, 1407 bp, synthetic construct
  misc_feature 1..1407: note = Heavy chain of antibody GA101 (HC 1329-2747)
  source 1..1407: mol_type = other DNA
atggagtttgggctgagctgggttttcctcgttgctctttttagaggtgtccagtgtcaggtgcagctggtgcagtctggggctgaggtgaagaagcctgggtcctccgtgaaggtctcctgcaaggcctctggatacgccttctcctactcttggatcaactgggtgcgacaggcccctggacaagggctcgagtggatgggacggatcttccctggcgatggtgacacagactacaacggcaagttcaagggcagagtcaccatcaccgccgacaagtccacgagcacagcctacatggaactgagcagcctgagatctgaggacacggccgtctattactgtgcacgaaacgtgttcgatggctattggctggtgtactggggccaggggaccctggtcaccgtcagcagcgctagcaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - DNA, 232 bp, synthetic construct
  misc_feature 1..232: note = Bovine Growth Hormone Polyadenylation signal (BGH pA)
  source 1..232: mol_type = other DNA
tgtgccttctaggcggccgcttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 11 - DNA, 17 bp, synthetic construct
  misc_feature 1..17: note = SV40 promoter
  source 1..17: mol_type = other DNA
ctgtggaatgtgtgtca

SEQ ID NO: 12 - DNA, 1122 bp, synthetic construct
  misc_feature 1..1122: note = Glutamine Synthetase (GS); Wild Type Glutamine Synthetase from CHO cell
  source 1..1122: mol_type = other DNA
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 13 - DNA, 130 bp, synthetic construct
  misc_feature 1..130: note = SV40 pA terminator sequence (SV40pA)
  source 1..130: mol_type = other DNA
acttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 14 - DNA, 7985 bp, synthetic construct
  misc_feature 1..7985: note = Tricistronic mAb GS
  source 1..7985: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattactcgagtccttagggagcgatccagcacgaggagaggccgggagggggcgggacggggcggggcctctgggagagtgggttgcggggaggctggcttttggcaggaagtaacgcatttgctggactcgagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctagcgtttaaacttaagcttggtaccgagctcggatccagaattcgccacaatggacatgagggtccctgctcagctcctggggctcctgctgctctggctctcaggtgccagatgtgacattgtaatgacacagaccccactctccctgcccgtcacccctggagagccggcctccatctcctgcaggtctagtaagagcctcctgcatagtaatggaatcacctatttgtactggtacctgcagaagccagggcagtctccacagctcctgatctatcagatgtctaatctggtgtccggggtccctgacaggttcagtggcagtggatcaggcacagattttacactgaaaatcagcagagtggaggctgaggatgttggggtttattactgcgcccaaaacctagagctgccctacactttcggcggagggaccaaggtggagatcaaacgtaccgtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaggatatcgtctagacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggagtttgggctgagctgggttttcctcgttgctctttttagaggtgtccagtgtcaggtgcagctggtgcagtctggggctgaggtgaagaagcctgggtcctccgtgaaggtctcctgcaaggcctctggatacgccttctcctactcttggatcaactgggtgcgacaggcccctggacaagggctcgagtggatgggacggatcttccctggcgatggtgacacagactacaacggcaagttcaagggcagagtcaccatcaccgccgacaagtccacgagcacagcctacatggaactgagcagcctgagatctgaggacacggccgtctattactgtgcacgaaacgtgttcgatggctattggctggtgtactggggccaggggaccctggtcaccgtcagcagcgctagcaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaatcgatgcctgcaggtggccggccacaccggtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataagcttgccacaaccccgggagatgaggatcgtttcgcatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggcgcgcctaatgagtgagctaactcacattaattgcgttgcgctcactgcccgctttccagtcgggaaacctgtcgtgccagctgcattaatgaatcggcgatcgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - DNA, 712 bp, synthetic construct
  misc_feature 1..712: note = CMV Promoter
  source 1..712: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattactcgagtccttagggagcgatccagcacgaggagaggccgggagggggcgggacggggcggggcctctgggagagtgggttgcggggaggctggcttttggcaggaagtaacgcatttgctggactcgagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 16 - DNA, 128 bp, synthetic construct
  misc_feature 1..128: note = Part of hamster?s Aprt (Adenine phosphoribosyltransferase) exon2 complementary strand
  source 1..128: mol_type = other DNA
tcgagtccttagggagcgatccagcacgaggagaggccgggagggggcgggacggggcggggcctctgggagagtgggttgcggggaggctggcttttggcaggaagtaacgcatttgctggactcga

SEQ ID NO: 17 - DNA, 41 bp, synthetic construct
  misc_feature 1..41: note = Multiple Cloning Site (MCS)
  source 1..41: mol_type = other DNA
gctagcgtttaaacttaagcttggtaccgagctcggatcca

SEQ ID NO: 18 - DNA, 729 bp, synthetic construct; identical to SEQ ID NO: 7
  misc_feature 1..729: note = Light chain of antibody GA101 (LC 1-729)
  source 1..729: mol_type = other DNA

SEQ ID NO: 19 - DNA, 20 bp, synthetic construct
  misc_feature 1..20: note = Wild type Internal Ribosome Entry Site
  source 1..20: mol_type = other DNA
atgataatatggccacaacc

SEQ ID NO: 20 - DNA, 1407 bp, synthetic construct; identical to SEQ ID NO: 9
  misc_feature 1..1407: note = Heavy chain of antibody GA101 (HC 1329-2747)
  source 1..1407: mol_type = other DNA

SEQ ID NO: 21 - DNA, 594 bp, synthetic construct
  misc_feature 1..594: note = Attenuated Internal Ribosome Entry Site (IRESatt)
  source 1..594: mol_type = other DNA
accggtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataagcttgccacaacc

SEQ ID NO: 22 - DNA, 23 bp, synthetic construct
  misc_feature 1..23: note = Attenuation stretch
  source 1..23: mol_type = other DNA
ccgggagatgaggatcgtttcgc

SEQ ID NO: 23 - DNA, 1122 bp, synthetic construct; identical to SEQ ID NO: 12
  misc_feature 1..1122: note = Glutamine Synthetase (GS); Wild Type Glutamine Synthetase from CHO cell
  source 1..1122: mol_type = other DNA

SEQ ID NO: 24 - DNA, 159 bp, synthetic construct
  misc_feature 1..159: note = pcDNA sequence between neo and SV40pA
  source 1..159: mol_type = other DNA
ttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccc

SEQ ID NO: 25 - DNA, 131 bp, synthetic construct
  misc_feature 1..131: note = SV40 pA terminator sequence (SV40pA)
  source 1..131: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 26 - DNA, 382 bp, synthetic construct
  misc_feature 1..382: note = Corresponding region with pcDNA3.1
  source 1..382: mol_type = other DNA
tataccgtcgacctctagctagagcttggcgtaatcatggtcatagctgtttcctgtgtgaaattgttatccgctcacaattccacacaacatacgagccggaagcataaagtgtaaagcctggcgcgcctaatgagtgagctaactcacattaattgcgttgcgctcactgcccgctttccagtcgggaaacctgtcgtgccagctgcattaatgaatcggcgatcgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaac

SEQ ID NO: 27 - DNA, 671 bp, synthetic construct
  misc_feature 1..671: note = pUC origin
  source 1..671: mol_type = other DNA
atgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggg

SEQ ID NO: 28 - DNA, 144 bp, synthetic construct
  misc_feature 1..144: note = Corresponding region with pcDNA3.1
  source 1..144: mol_type = other DNA
gtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacag

SEQ ID NO: 29 - DNA, 861 bp, synthetic construct
  misc_feature 1..861: note = Ampicillin (Amp) resistance gene on complementary strand
  source 1..861: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 30 - DNA, 136 bp, synthetic construct
  misc_feature 1..136: note = beta-Lactamase (bla) promoter
  source 1..136: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 31 - DNA, 57 bp, synthetic construct
  misc_feature 1..57: note = primer GSsmaUP
  source 1..57: mol_type = other DNA
gtgtgacccgggagatgaggatcgtttcgcatggccacctcagcaagttcccacttg

SEQ ID NO: 32 - DNA, 42 bp, synthetic construct
  misc_feature 1..42: note = primer GSBstBILP
  source 1..42: mol_type = other DNA
gaattcttcgaattagtttttgtattcgaagggctcgtcgcc

SEQ ID NO: 33 - DNA, 96 bp, synthetic construct
  misc_feature 1..96: note = primer GSSMASV4UP
  source 1..96: mol_type = other DNA
gtgctcccgggagcttgtatatccattttcggatctgatcaagagacaggatgaggatcggccaccatggccacctcagcaagttcccacttgaac

SEQ ID NO: 34 - DNA, 54 bp, synthetic construct
  misc_feature 1..54: note = primer GSBSTBISV4LP
  source 1..54: mol_type = other DNA
ggtcatttcgaaccccagagtcccgcttagtttttgtattggaagggctcgtcg

SEQ ID NO: 35 - DNA, 36 bp, synthetic construct
  misc_feature 1..36: note = primer R324CFP
  source 1..36: mol_type = other DNA
cgcagtgccagcatctgcattccccggactgtcggc

SEQ ID NO: 36 - DNA, 36 bp, synthetic construct
  misc_feature 1..36: note = primer R324CLP
  source 1..36: mol_type = other DNA
gccgacagtccggggaatgcagatgctggcactgcg

SEQ ID NO: 37 - DNA, 39 bp, synthetic construct
  misc_feature 1..39: note = primer R341CFP
  source 1..39: mol_type = other DNA
ggttactttgaagaccgctgcccctctgccaattgtgac

SEQ ID NO: 38 - DNA, 39 bp, synthetic construct
  misc_feature 1..39: note = primer R341CLP
  source 1..39: mol_type = other DNA
gtcacaattggcagaggggcagcggtcttcaaagtaacc

SEQ ID NO: 39 - DNA, 33 bp, synthetic construct
  misc_feature 1..33: note = primer R324AUP
  source 1..33: mol_type = other DNA
cgcagtgccagcatcgccattccccggactgtc

SEQ ID NO: 40 - DNA, 33 bp, synthetic construct
  misc_feature 1..33: note = primer R324ALP
  source 1..33: mol_type = other DNA
gacagtccggggaatggcgatgctggcactgcg

SEQ ID NO: 41 - DNA, 33 bp, synthetic construct
  misc_feature 1..33: note = primer R341AUP
  source 1..33: mol_type = other DNA
tactttgaagaccgcgccccctctgccaattgt

SEQ ID NO: 42 - DNA, 33 bp, synthetic construct
  misc_feature 1..33: note = primer R341ALP
  source 1..33: mol_type = other DNA
acaattggcagagggggcgcggtcttcaaagta

SEQ ID NO: 43 - DNA, 36 bp, synthetic construct
  misc_feature 1..36: note = primer GSM1 UP
  source 1..36: mol_type = other DNA
cctgagtggaattttgctggctctagtacctttcag

SEQ ID NO: 44 - DNA, 36 bp, synthetic construct
  misc_feature 1..36: note = primer GSM1 LP
  source 1..36: mol_type = other DNA
ctgaaaggtactagtgccagcaaaattccactcagg

SEQ ID NO: 45 - DNA, 39 bp, synthetic construct
  misc_feature 1..39: note = primer GSM2 UP
  source 1..39: mol_type = other DNA
tggaattttgatggctctgctacctttcagtctgagggc

SEQ ID NO: 46 - DNA, 39 bp, synthetic construct
  misc_feature 1..39: note = primer GSM2 LP
  source 1..39: mol_type = other DNA
gccctcagactgaaaggtagcagagccatcaaaattcca

SEQ ID NO: 47 - DNA, 36 bp, synthetic construct
  misc_feature 1..36: note = primer GSM4 UP
  source 1..36: mol_type = other DNA
cacccctggtttggaatggcacaggagtatactctg

SEQ ID NO: 48 - DNA, 36 bp, synthetic construct
  misc_feature 1..36: note = primer GSM4 LP
  source 1..36: mol_type = other DNA
cagagtatactcctgtgccattccaaaccaggggtg

SEQ ID NO: 49 - DNA, 39 bp, synthetic construct
  misc_feature 1..39: note = primer GSM5 UP
  source 1..39: mol_type = other DNA
tggtttggaatggaacaggcgtatactctgatgggaaca

SEQ ID NO: 50 - DNA, 39 bp, synthetic construct
  misc_feature 1..39: note = primer GSM5 LP
  source 1..39: mol_type = other DNA
tgttcccatcagagtatacgcctgttccattccaaacca

SEQ ID NO: 51 - DNA, 36 bp, synthetic construct
  misc_feature 1..36: note = primer GSM6 UP
  source 1..36: mol_type = other DNA
ccccaaggtccgtatgcctgtggtgtgggcgcagac

SEQ ID NO: 52 - DNA, 36 bp, synthetic construct
  misc_feature 1..36: note = primer GSM6 LP
  source 1..36: mol_type = other DNA
gtctgcgcccacaccacaggcatacggaccttgggg

SEQ ID NO: 53 - DNA, 33 bp, synthetic construct
  misc_feature 1..33: note = primer GSM8 UP
  source 1..33: mol_type = other DNA
ggggtcaagattacagcaacaaatgctgaggtc

SEQ ID NO: 54 - DNA, 33 bp, synthetic construct
  misc_feature 1..33: note = primer GSM8 LP
  source 1..33: mol_type = other DNA
gacctcagcatttgttgctgtaatcttgacccc

SEQ ID NO: 55 - DNA, 33 bp, synthetic construct
  misc_feature 1..33: note = primer GSM9 UP
  source 1..33: mol_type = other DNA
acaggaacaaatgctgcggtcatgcctgcccag

SEQ ID NO: 56 - DNA, 33 bp, synthetic construct
  misc_feature 1..33: note = primer GSM9 LP
  source 1..33: mol_type = other DNA
ctgggcaggcatgaccgcagcatttgttcctgt

SEQ ID NO: 57 - DNA, 33 bp, synthetic construct
  misc_feature 1..33: note = primer GSM10 UP
  source 1..33: mol_type = other DNA
atgcctgcccagtgggcattccaaataggaccc

SEQ ID NO: 58 - DNA, 33 bp, synthetic construct
  misc_feature 1..33: note = primer GSM10 LP
  source 1..33: mol_type = other DNA
gggtcctatttggaatgcccactgggcaggcat

SEQ ID NO: 59 - DNA, 36 bp, synthetic construct
  misc_feature 1..36: note = primer GSM12 UP
  source 1..36: mol_type = other DNA
attcctgggaactgggcaggtgcaggctgccatacc

SEQ ID NO: 60 - DNA, 36 bp, synthetic construct
  misc_feature 1..36: note = primer GSM12 LP
  source 1..36: mol_type = other DNA
ggtatggcagcctgcacctgcccagttcccaggaat

SEQ ID NO: 61 - DNA, 33 bp, synthetic construct
  misc_feature 1..33: note = primer GSM13 UP
  source 1..33: mol_type = other DNA
cctgggaactggaatgctgcaggctgccatacc

SEQ ID NO: 62 - DNA, 33 bp, synthetic construct
  misc_feature 1..33: note = primer GSM13 LP
  source 1..33: mol_type = other DNA
ggtatggcagcctgcagcattccagttcccagg

SEQ ID NO: 63 - DNA, 33 bp, synthetic construct
  misc_feature 1..33: note = primer GSM14 UP
  source 1..33: mol_type = other DNA
aatggtgcaggctgcgcaaccaactttagcacc

SEQ ID NO: 64 - DNA, 33 bp, synthetic construct
  misc_feature 1..33: note = primer GSM14 LP
  source 1..33: mol_type = other DNA
ggtgctaaagttggttgcgcagcctgcaccatt

SEQ ID NO: 65 - DNA, 33 bp, synthetic construct
  misc_feature 1..33: note = primer GSM15 UP
  source 1..33: mol_type = other DNA
gcaggctgccataccgcatttagcaccaaggcc

SEQ ID NO: 66 - DNA, 33 bp, synthetic construct
  misc_feature 1..33: note = primer GSM15 LP
  source 1..33: mol_type = other DNA
ggccttggtgctaaatgcggtatggcagcctgc

SEQ ID NO: 67 - DNA, 36 bp, synthetic construct
  misc_feature 1..36: note = primer GSM16 UP
  source 1..36: mol_type = other DNA
ggctgccataccaactttgcaaccaaggccatgcgg

SEQ ID NO: 68 - DNA, 36 bp, synthetic construct
  misc_feature 1..36: note = primer GSM16 LP
  source 1..36: mol_type = other DNA
ccgcatggccttggttgcaaagttggtatggcagcc

SEQ ID NO: 69 - DNA, 33 bp, synthetic construct
  misc_feature 1..33: note = primer GSM17 UP
  source 1..33: mol_type = other DNA
agcaccaaggccatggcggaggagaatggtctg

SEQ ID NO: 70 - DNA, 33 bp, synthetic construct
  misc_feature 1..33: note = primer GSM17 LP
  source 1..33: mol_type = other DNA
cagaccattctcctccgccatggccttggtgct

SEQ ID NO: 71 - DNA, 33 bp, synthetic construct
  misc_feature 1..33: note = primer GSM18 UP
  source 1..33: mol_type = other DNA
ctggacaatgcccgtgctctgactgggttccac

SEQ ID NO: 72 - DNA, 33 bp, synthetic construct
  misc_feature 1..33: note = primer GSM18 LP
  source 1..33: mol_type = other DNA
gtggaacccagtcagagcacgggcattgtccag

SEQ ID NO: 73 - DNA, 33 bp, synthetic construct
  misc_feature 1..33: note = primer GSM19 UP
  source 1..33: mol_type = other DNA
ctgactgggttccacgcaacgtccaacatcaac

SEQ ID NO: 74 - DNA, 33 bp, synthetic construct
  misc_feature 1..33: note = primer GSM19 LP
  source 1..33: mol_type = other DNA
gttgatgttggacgttgcgtggaacccagtcag

SEQ ID NO: 75 - DNA, 33 bp, synthetic construct
  misc_feature 1..33: note = primer GSM20 UP
  source 1..33: mol_type = other DNA
gctggtgtcgccaatgccagtgccagcatccgc

SEQ ID NO: 76 - DNA, 33 bp, synthetic construct
  misc_feature 1..33: note = primer GSM20 LP
  source 1..33: mol_type = other DNA
gcggatgctggcactggcattggcgacaccagc

SEQ ID NO: 77 - DNA, 33 bp, synthetic construct
  misc_feature 1..33: note = primer GSM21 UP
  source 1..33: mol_type = other DNA
caggagaagaaaggtgcttttgaagaccgccgc

SEQ ID NO: 78 - DNA, 33 bp, synthetic construct
  misc_feature 1..33: note = primer GSM21 LP
  source 1..33: mol_type = other DNA
gcggcggtcttcaaaagcacctttcttctcctg

SEQ ID NO: 79 - DNA, 36 bp, synthetic construct
  misc_feature 1..36: note = primer GSM22 UP
  source 1..36: mol_type = other DNA
aagaaaggttactttgcagaccgccgcccctctgcc

SEQ ID NO: 80 - DNA, 36 bp, synthetic construct
  misc_feature 1..36: note = primer GSM22 LP
  source 1..36: mol_type = other DNA
ggcagaggggcggcggtctgcaaagtaacctttctt

SEQ ID NO: 81 - DNA, 33 bp, synthetic construct
  misc_feature 1..33: note = primer GSM23 UP
  source 1..33: mol_type = other DNA
actgtcggccaggaggcgaaaggttactttgaa

SEQ ID NO: 82 - DNA, 33 bp, synthetic construct
  misc_feature 1..33: note = primer GSM23 LP
  source 1..33: mol_type = other DNA
ttcaaagtaacctttcgcctcctggccgacagt

SEQ ID NO: 83 - DNA, 33 bp, synthetic construct
  misc_feature 1..33: note = primer GSM24 UP
  source 1..33: mol_type = other DNA
ggttactttgaagacgcccgcccctctgccaat

SEQ ID NO: 84 - DNA, 33 bp, synthetic construct
  misc_feature 1..33: note = primer GSM24 LP
  source 1..33: mol_type = other DNA
attggcagaggggcgggcgtcttcaaagtaacc

SEQ ID NO: 85 - DNA, 7985 bp, synthetic construct
  misc_feature 1..7985: note = Tricistronic GA101 GS R324C
  source 1..7985: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattactcgagtccttagggagcgatccagcacgaggagaggccgggagggggcgggacggggcggggcctctgggagagtgggttgcggggaggctggcttttggcaggaagtaacgcatttgctggactcgagtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctagcgtttaaacttaagcttggtaccgagctcggatccagaattcgccacaatggacatgagggtccctgctcagctcctggggctcctgctgctctggctctcaggtgccagatgtgacattgtaatgacacagaccccactctccctgcccgtcacccctggagagccggcctccatctcctgcaggtctagtaagagcctcctgcatagtaatggaatcacctatttgtactggtacctgcagaagccagggcagtctccacagctcctgatctatcagatgtctaatctggtgtccggggtccctgacaggttcagtggcagtggatcaggcacagattttacactgaaaatcagcagagtggaggctgaggatgttggggtttattactgcgcccaaaacctagagctgccctacactttcggcggagggaccaaggtggagatcaaacgtaccgtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaggatatcgtctagacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggagtttgggctgagctgggttttcctcgttgctctttttagaggtgtccagtgtcaggtgcagctggtgcagtctggggctgaggtgaagaagcctgggtcctccgtgaaggtctcctgcaaggcctctggatacgccttctcctactcttggatcaactgggtgcgacaggcccctggacaagggctcgagtggatgggacggatcttccctggcgatggtgacacagactacaacggcaagttcaagggcagagtcaccatcaccgccgacaagtccacgagcacagcctacatggaactgagcagcctgagatctgaggacacggccgtctattactgtgcacgaaacgtgttcgatggctattggctggtgtactggggccaggggaccctggtcaccgtcagcagcgctagcaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaatcgatgcctgcaggtggccggccacaccggtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataagcttgccacaaccccgggagatgaggatcgtttcgcatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatctgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggcgcgcctaatgagtgagctaactcacattaattgcgttgcgctcactgcccgctttccagtcgggaaacctgtcgtgccagctgcattaatgaatcggcgatcgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 86 - DNA, 128 bp, synthetic construct; identical to SEQ ID NO: 16
  misc_feature 1..128: note = Part of hamster?s Aprt (Adenine phosphoribosyltransferase) exon2 complementary strand
  source 1..128: mol_type = other DNA

SEQ ID NO: 87 - DNA, 205 bp, synthetic construct
  misc_feature 1..205: note = Second portion of CMV promoter
  source 1..205: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 88 - DNA, 41 bp, synthetic construct; identical to SEQ ID NO: 17
  misc_feature 1..41: note = Multiple Cloning Site (MCS)
  source 1..41: mol_type = other DNA

SEQ ID NO: 89 - DNA, 729 bp, synthetic construct; identical to SEQ ID NO: 7
  misc_feature 1..729: note = Light chain of antibody GA101 (LC 1-729)
  source 1..729: mol_type = other DNA

SEQ ID NO: 90 - DNA, 20 bp, synthetic construct
  misc_feature 1..20: note = Wild type Internal Ribosome Entry Site (IRES)
  source 1..20: mol_type = other DNA
atgataatatggccacaacc

SEQ ID NO: 91 - DNA, 1407 bp, synthetic construct; identical to SEQ ID NO: 9
  misc_feature 1..1407: note = Heavy chain of antibody GA101 (HC 1329-2747)
  source 1..1407: mol_type = other DNA

SEQ ID NO: 92 - DNA, 594 bp, synthetic construct; identical to SEQ ID NO: 21
  misc_feature 1..594: note = Attenuated Internal Ribosome Entry Site (attIRES)
  source 1..594: mol_type = other DNA

SEQ ID NO: 93 - DNA, 23 bp, synthetic construct
  misc_feature 1..23: note = Attenuation stretch
  source 1..23: mol_type = other DNA
ccgggagatgaggatcgtttcgc

SEQ ID NO: 94 - DNA, 1122 bp, synthetic construct
  misc_feature 1..1122: note = Glutamine Synthetase (GS) having R324C mutation
  source 1..1122: mol_type = other DNA
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaattccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatctgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 95 - DNA, 159 bp, synthetic construct; identical to SEQ ID NO: 24
  misc_feature 1..159: note = pcDNA sequence between neo and SV40pA
  source 1..159: mol_type = other DNA

SEQ ID NO: 96 - DNA, 131 bp, synthetic construct; identical to SEQ ID NO: 25
  misc_feature 1..131: note = SV40 pA terminator sequence (SV40pA)
  source 1..131: mol_type = other DNA

SEQ ID NO: 97 - DNA, 382 bp, synthetic construct; identical to SEQ ID NO: 26
  misc_feature 1..382: note = Corresponding region with pcDNA3.1
  source 1..382: mol_type = other DNA

SEQ ID NO: 98 - DNA, 671 bp, synthetic construct; identical to SEQ ID NO: 27
  misc_feature 1..671: note = PUC origin
  source 1..671: mol_type = other DNA

SEQ ID NO: 99 - DNA, 144 bp, synthetic construct; identical to SEQ ID NO: 28
  misc_feature 1..144: note = Corresponding region with pcDNA3.1
  source 1..144: mol_type = other DNA

SEQ ID NO: 100 - DNA, 861 bp, synthetic construct; identical to SEQ ID NO: 29
  misc_feature 1..861: note = Ampicillin (Amp) resistance gene on complementary strand
  source 1..861: mol_type = other DNA

SEQ ID NO: 101 - DNA, 136 bp, synthetic construct; identical to SEQ ID NO: 30
  misc_feature 1..136: note = beta-Lactamase (bla) promoter
  source 1..136: mol_type = other DNA

SEQ ID NO: 102 - DNA, 1122 bp, Cricetulus griseus
  source 1..1122: mol_type = unassigned DNA
atggccacctcagcaagttcccacttgaacaaaggcatcaagcaaatgtacatgtccctgccccagggtgagaaagtccaagccatgtatatctgggttgatggtaccggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcaaagagcccaacaagctggtgttctgtgaagtcttcaagtacaaccggaagcctgcagagaccaatttaagacacacgtgtaaacggataatggacatggtgagcaaccagcacccctggtttggaatggaacaggagtatactctcttgggaacagatgggcacccttttggttggccttccgatggcttccctgggccccaaggtctgtattactgtggtgtgggcgcagacaaagcctatcgcagggatatcatggaggctcactaccttgcctgcttgtatgctggggtcaagattacaggaacatatgctgaggtcaagcatgcccagtgggaattccaaataggaccctgtgaaggaatccgcatgggagatcatctgtgggtggcccgtttcatcttgcatcgagtatgtaaagactttggggtaatagcaacctttgaccccaagcccattcctgggaactggaatggtgcaggctgccataccaactttagtaccaagaccatgcgggaggagaatggtctgaagcacatcaaggaggccattgagaaactaagcaagcggcaccggtaccatattcgagcctacgatcccaagggggggctggacaatgcccgtcgtctgactgggttccacaaaacgtccaacatcaacgacttttcagctggcgtcgccgatcgcagtgccagcatccgcattccccggactgtcggccaggagaagaaaggttactttgaagcccgctgcccctctgccaattgtgacccctttgcagtgacagaagccatcgtccgcacatgccttctcaatgagactggcgacgagcccttccaatacaaaaactaa

SEQ ID NO: 103 - protein, 373 aa, Cricetulus griseus
MATSASSHLNKGIKQMYMSLPQGEKVQAMYIWVDGTGEGLRCKTRTLDCEPKCVEELPEWNFDGSSTFQSEGSNSDMYLSPVAMFRDPFRKEPNKLVFCEVFKYNRKPAETNLRHTCKRIMDMVSNQHPWFGMEQEYTLLGTDGHPFGWPSDGFPGPQGLYYCGVGADKAYRRDIMEAHYLACLYAGVKITGTYAEVKHAQWEFQIGPCEGIRMGDHLWVARFILHRVCKDFGVIATFDPKPIPGNWNGAGCHTNFSTKTMREENGLKHIKEAIEKLSKRHRYHIRAYDPKGGLDNARRLTGFHKTSNINDFSAGVADRSASIRIPRTVGQEKKGYFEARCPSANCDPFAVTEAIVRTCLLNETGDEPFQYKN

(19) United States
(12) Patent Application Publication
(10) Pub. No.: US 2024/0124868 A1
(43) Pub. Date: Apr. 18, 2024

(54) ATTENUATED GLUTAMINE SYNTHETASE AS A SELECTION MARKER

(71) Applicant: AGENCY FOR SCIENCE, TECHNOLOGY AND RESEARCH, Singapore (SG)

(72) Inventors: Pao Chun Lin, Singapore (SG); Zhiwei Song, Singapore (SG)

(73) Assignee: AGENCY FOR SCIENCE, TECHNOLOGY AND RESEARCH, Singapore (SG)

(21) Appl. No.: 18/493,395

(22) Filed: Oct. 24, 2023

Related U.S. Application Data
(62) Division of application No. 16/461,320, filed on May 15, 2019.

(30) Foreign Application Priority Data
Nov. 16, 2016 (SG) 10201609619S

(51) Int. Cl. C12N 9/00; C12N 15/52; C12N 15/85

(52) U.S. Cl. CPC C12N 9/93; C12N 15/52; C12N 15/85; C12N 2840/203; C12Y 603/01002

(57) ABSTRACT

Disclosed is an expression vector comprising a polynucleotide encoding for a glutamine synthetase with reduced activity compared to a wild type glutamine synthetase. Also disclosed are host cells, methods for preparing stable cell line, methods of producing polypeptide of interest, and kits thereof.

### CROSS-REFERENCE TO RELATED APPLICATIONS

This application is a Divisional of Ser. No. 16/461,320, filed May 15, 2019, which is a National Stage entry of PCT/SG2017/050570, filed Nov. 16, 2017, which claims the benefit of priority of Singapore provisional application No. 10201609619S, filed 16 Nov. 2016, the contents of which are hereby incorporated by reference in its entirety for all purposes.

### SEQUENCE LISTING

The instant application contains a Sequence Listing which has been submitted electronically in XML format and is hereby incorporated by reference in its entirety. Said XML copy, created Oct. 24, 2023 is named 56052-906N01US-ST26.xml and is 171 KB in size.

### FIELD OF THE INVENTION

The present invention generally relates to mutation or genetic engineering. In particular, the present invention relates to expression vectors and methods for selecting the vector-containing host using markers.

### BACKGROUND OF THE INVENTION

In the field of stable cell line development for biologic production, there is a need to select for cells that are high producers and yet stable at the same time. An example of a selection system used in the art is glutamine synthetase (GS) selection system. In certain cells typically used for stable cell line development, the glutamine synthetase (GS) has been inactivated. As such, these cells cannot synthesize glutamine on their own and can only survive and grow if glutamine is added into the culture medium. Therefore, the GS gene has been widely used in expression vectors as a positive selection marker. Thus, only cells that have incorporated expression vectors comprising GS gene will survive when glutamine is removed from the culture medium. Additionally, besides removing glutamine from the culture medium, to ensure that only high producer cells survive, a GS inhibitor will also be added. Addition of a GS inhibitor may amplify the copy number of the expression cassette in the chromosome because GS activity is inhibited. Thus, only the cells with higher copy number of GS gene can survive a GS inhibitor treatment.

Using the method described above, in order to ultimately obtain cell lines that are stable and high producers, hundreds of single clones need to be isolated to determine their productivity. Their stability upon a GS inhibitor removal also has to be assessed over at least 60 generations. This is a labor intensive and time consuming process.

In view of the above, in order to streamline the process of generating stable cell line, there is a need to provide an alternative expression vector comprising a polynucleotide encoding for an alternative glutamine synthetase.

### SUMMARY OF THE INVENTION

In one aspect, there is provided an expression vector comprising a polynucleotide encoding for a glutamine synthetase with reduced activity compared to a wild type glutamine synthetase. In one embodiment, the expression vector further comprising at least one polynucleotide encoding for a polypeptide of interest. In another embodiment, the glutamine synthetase with reduced activity comprises one or more mutations as compared to a wild type glutamine synthetase. In yet another embodiment, the glutamine synthetase with reduced activity comprises one or more mutations in the glutamate binding region, the ATP binding region and/or the ammonia binding region. In yet another embodiment, (i) the mutation in the glutamate binding region is of at least one amino acid at the position selected from the group consisting of 134, 136, 196, 203, 248, 249, 253, 299, 319, 338, and 340 of a glutamine synthetase; or (ii) the mutation in the ammonia binding region is of at least one amino acid at the position selected from the group consisting of 63, 66, 162, and 305 of a glutamine synthetase; or (iii) the mutation in the ATP binding region is of at least one amino acid at the position selected from the group consisting of 192, 255, 257, 262, 333, 336, and 324 of a glutamine synthetase; wherein the glutamine synthetase has the amino acid sequence shown in SEQ ID NO: 1, SEQ ID NO: 2, SEQ ID NO: 3, or SEQ ID NO:4. In yet another embodiment, the mutation is of at least one amino acid at the position selected from the group consisting of 12, 19, 63, 66, 91, 116, 134, 136, 160, 162, 172, 176, 181, 192, 194, 196, 198, 199, 203, 230, 248, 249, 253, 255, 257, 260, 262, 271, 299, 305, 319, 336, 338, 333, 340, 324 and 341 of a glutamine synthetase, wherein the glutamine synthetase has the amino acid sequence shown in SEQ ID NO: 1, SEQ ID NO: 2, SEQ ID NO: 3, or SEQ ID NO:4. In yet another embodiment, the mutation is a substitution. In yet another embodiment, the mutation in the glutamine synthetase is selected from the group consisting of R324C, R341C, D63A, S66A, E134A, E136A, Y162A, G192A, E196A, E203A, N248A, G249A, H253A, N255A, S257A, R262A, R299A, E305A, R319A, R324A, Y336A, E338A, K333A, R340A, and R341A of a glutamine synthetase, wherein the glutamine synthetase has the amino acid sequence shown in SEQ ID NO: 1, SEQ ID NO: 2, SEQ ID NO: 3, or SEQ ID NO:4.

In yet another embodiment, the polynucleotide encoding for a glutamine synthetase with reduced activity is operatively linked to a promoter. In yet another embodiment, only one of the at least one polynucleotide encoding for a polypeptide of interest is operatively linked to a promoter. In yet another embodiment, the polynucleotide encoding for a glutamine synthetase with reduced activity compared to a wild type glutamine synthetase is not linked to a promoter. In yet another embodiment, the polypeptide of interest is selected from the group consisting of a recombinant protein and a part thereof, a fusion protein and a part thereof, an antibody and a part thereof. In yet another embodiment, the polypeptide of interest is the heavy chain of an antibody of interest and/or light chain of an antibody of interest. In yet another embodiment, the expression vector comprises two polynucleotides each encoding for a polypeptide of interest, wherein one of the polynucleotides encodes for the light chain of an antibody of interest, and one of the polynucleotides encodes for the heavy chain of the same antibody of interest. In yet another embodiment, the polynucleotide encoding for the light chain of an antibody of interest and the polynucleotide encoding for the heavy chain of the same antibody of interest are separated by polynucleotides encoding an internal ribosome entry site (IRES). In yet another embodiment, the polynucleotide encoding for the heavy chain of an antibody of interest is separated from the polynucleotide encoding for a glutamine synthetase with reduced activity by polynucleotide encoding an internal ribosome entry site (IRES). In yet another embodiment, the polynucleotide encoding the internal ribosome entry site separating the polynucleotide encoding for the light chain of an antibody of interest and the polynucleotide encoding for the heavy chain of the same antibody of interest is polynucleotide encoding a wild type internal ribosome entry site (IRES), and wherein the internal ribosome entry site separating the polynucleotide encoding for the heavy chain of an antibody of interest from the polynucleotide encoding for a glutamine synthetase with reduced activity is polynucleotide encoding an internal ribosome entry site with attenuated translation efficiency (IRESatt).

In another aspect, there is provided a host cell comprising the expression vector as described herein. In yet another aspect, there is provided a method for preparing stable cell line, comprising: (a) transforming a host cell having no glutamine synthetase activity with the expression vector as described herein; and (b) culturing the transformed host cell in a medium that selectively allows the proliferation of the transformed host cells comprising an amplified number of copies of the vector to be selected.

In yet another aspect, there is provided a method of producing polypeptide of interest, comprising: (a) transforming a host cell having no glutamine synthetase activity with the expression vector as described herein; (b) culturing the transformed host cell in a medium that selectively allows the production of polypeptide of interest; and (c) collecting the polypeptide of interest from the medium of (b). In yet another embodiment, the medium is a glutamine free cell culture medium. In yet another embodiment, the method does not comprise the use of a glutamine synthetase inhibitor, optionally wherein the glutamine synthetase inhibitor is methionine sulphoximine (MSX).

In yet another aspect, there is provided a kit comprising the expression vector as described herein. In yet another embodiment, the kit further comprises at least one of the following: (i) a host cell having no glutamine synthetase activity compared to a wild type cell; and (ii) a transfection medium or means to carry out a transfection. In yet another embodiment, the kit further comprises a glutamine free cell culture medium.

### BRIEF DESCRIPTION OF THE DRAWINGS

The invention will be better understood with reference to the detailed description when considered in conjunction with the non-limiting examples and the accompanying drawings, in which:

FIGS. 1A-1B show a pair of line graphs depicting amount of antibodies produced using CHOK1 GFT^{-/-} GS^{-/-} cells that are transfected with either Tricistronic mAb-GS (SEQ ID NO: 14) or Tricistronic mAb-GSatt (R324C) (SEQ ID NO: 85) in minipools format. FIG. 1A depicts the experimental result for Antibody A and FIG. 1B depicts the experimental result for Antibody B. Thus, FIGS. 1A-1B illustrate that for production of both Antibody A and Antibody B, the amount of antibody produced using Tricistronic mAb-GSatt (R324C) is higher than the amount of antibody produced using Tricistronic mAb-GS.

FIGS. 2A-2B show a pair of bar graphs depicting amount of antibodies titer (mAb titer) produced using CHOK1 GFT−/− GS−/− cells that are transfected with either Tricistronic mAb-GS (SEQ ID NO: 14) or Tricistronic mAb-GSatt (R324C) (SEQ ID NO: 85) in stable suspension pool format. For preliminary analysis, the WT GS system (i.e. CHOK1 GFT−/− GS−/− cells that are transfected with Tricistronic mAb-GS) and attenuated GS system (i.e. CHOK1 GFT−/− GS−/− cells that are transfected with mAb-GSatt (R324C)) were compared directly in the absence of glutamine for a fair comparison. Thus, the WT GS pools were not treated with MSX. FIG. 2A depicts the experimental result for Antibody A; demonstrating that amount of mAb titer produced using Tricistronic mAb-GSatt (R324C) (data labeled as Mut1, Mut2, and Mut3) is approximately 9.9-fold more than amount of mAb titer produced using Tricistronic mAb-GS (data labeled as WT1, WT2, and WT3). FIG. 2B depicts the experimental result for Antibody B; demonstrating that amount of mAb titer produced using Tricistronic mAb-GSatt (R324C) (data labeled as Mut1) is approximately 2.6-fold more than amount of mAb titer produced using Tricistronic mAb-GS (data labeled as WT1, WT2, and WT3). Thus, FIGS. 2A-2B illustrate that amount of mAb titer produced using Tricistronic mAb-GSatt (R324C) is higher than amount of mAb titer produced using Tricistronic mAb-GS.

FIG. 3 shows a scatter plot depicting the amount of antibodies titer (mAb titer) produced by stable pools using (a) bicistronic expression vector having wild type GS (WT) as a selection marker (data points are shown in diamonds), or (b) bicistronic expression vector having wild type GS (WT) as a selection marker and wherein the cells are also treated with 25 μM of methionine sulphoximine (MSX) (data points are shown in squares), or (c) bicistronic expression vector having attenuated GS (GSatt) as a selection marker (cells are not treated with MSX; data points are shown in triangles). Thus, FIG. 3 illustrates that batch culture mAb titer in stable pools transfected with the mutant GS (i.e. bicistronic mAb-GSatt (R324C)) are generally higher than those WT GS pools (regardless whether the WT GS pools is treated with MSX (25 μM) or not).

FIG. 4 shows a bar graph depicting the comparison of the GS activities of WT GS and several GS mutants. FIG. 4 depicts the GS activities of WT GS and GS mutants that have been blanked using the cell lysate of untransfected CHO GS^{-/-} cells and expression normalized using their luciferase activity. The GS activities of GS mutants in FIG. 4 are represented as fold-change over the GS activity of WT GS. Thus, FIG. 4 shows that several sites on the GS, when mutated from any amino acid to alanine, would attenuate the GS activity.

FIG. 5 shows a bar graph depicting the stability assessment of 20 single clones derived from 2 stable pools (top: pool 1, bottom: pool 2) transfected with Tricistronic mAb-GSatt (R324C) (SEQ ID NO: 85). Thus, FIG. 5 illustrates that almost all the single clones (more than 95%) are above the stability level of 70%, which signifies that more than 95% of the single clones are stable.

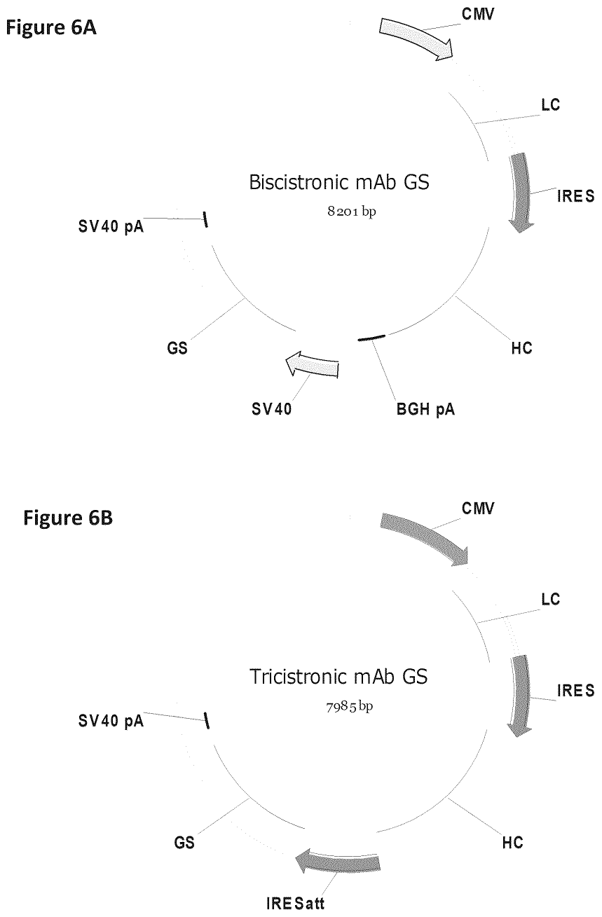

FIGS. 6A-6C show graphical illustrations depicting expression vector maps. FIG. 6A depicts the expression vector map of bicistronic mAb GS. The full length sequence that corresponds to bicistronic mAb GS is listed as SEQ ID NO: 5. Sequences that correspond to specific regions of the expression vector map of bicistronic mAb GS are listed as SEQ ID NOs: 6 to 13. FIG. 6B depicts the expression vector map of tricistronic mAb GS. The full length sequence that corresponds to tricistronic mAb GS is listed as SEQ ID NO: 14. Sequences that correspond to specific regions of the expression vector map of tricistronic mAb GS are listed as SEQ ID NOs: 15 to 30. FIG. 6C depicts the expression vector map of tricistronic mAb GS R324C, an exemplary expression vector that express attenuated glutamine synthetase. The full length sequence that corresponds to tricistronic mAb GS R324C is listed as SEQ ID NO: 85. Sequences that correspond to specific regions of the expression vector map of tricistronic mAb GS R324C are listed as SEQ ID NOs: 86 to 101. Thus, FIGS. 6A-6C show examples of the map of expression vector as described herein.

FIG. 7 shows a sequence alignment comparing GS protein sequences from four different species, namely Chinese hamster (labeled as “XP_003502909-CHO”; SEQ ID NO: 2), mouse (labeled as “NP_032157-mouse”; SEQ ID NO: 3), rat (labeled as “NP_058769-rat”; SEQ ID NO: 4), and human (labeled as “NP_0025056-human”; SEQ ID NO: 1). The line titled “consensus” shows the amino acid residues that are conserved among all four of the species. The arrows indicate the location of the point mutation that will be introduced to the WT GS. The type point mutation for each number is listed on Table 1. The dotted arrows indicate the location of the known human congenital disease mutations. Thus, FIG. 7 shows that the GS protein sequences among the four species listed are highly conserved. The percent sequence similarity of the GS protein sequences of Chinese hamster to mouse is 97%, the percent sequence similarity of the GS protein sequences of Chinese hamster to rat is 96%, and the percent sequence similarity of the GS protein sequences of Chinese hamster to human is 94%.

FIG. 8 shows a combined scatter plot and table depicting the comparisons of the amount of mAb titers generated using WT GS and GS mutants as selection markers in CHO-K1 GS^{-/-} cells. For the WT samples, samples labeled as “GSwt” were not subjected to MSX selection while samples labeled as “GSwt+MSX” were subjected to 25 μM MSX selection. For the GS mutant samples, “GSm1” corresponds to GS having D63A mutation site, “GSm12” corresponds to GS having N248A mutation site, “GSm16” corresponds to GS having S257A mutation site, “GSm19” corresponds to GS having E305A mutation site, and “R324C” corresponds to GS having R324C mutation site. Thus, FIG. 8 illustrates that the average mAb titers generated using GS mutants—Gsm1, Gsm19 and R324C as selection markers are higher than the average mAb titers generated using WT GS as selection markers.

FIGS. 9A-9B show a pair of bar graphs depicting the stability assessment of five random clones from stable generated using GSwt as the selection marker (as shown on FIG. 9A) or GSm16 (i.e. GS having S257A mutation site) as the selection marker (as shown on FIG. 9B). Thus, FIGS. 9A-9B show that only two out of five GSwt clones were stable (i.e. maintained at least 70% of its original titer level) whereas four out of five GSm16 clones were stable.

FIG. 10 shows a sequence alignment comparing wild type GS protein sequences from CHO cells (labeled as “GS”; SEQ ID NO: 2) and pseudoGS protein sequences from CHO cells (labeled as “psGS”; SEQ ID NO: 103). PseudoGS (psGS) is a stretch of genomic DNA which seems to also code for the GS protein. This psGS genomic locus, unlike the wild type GS gene, does not contain any intervening intron sequences. PseudoGS protein is typically not expressed. PseudoGS comprises several mutations throughout the protein as shown (using black arrows) in the alignment. Importantly, psGS protein comprises 2 mutation sites that are identified to have attenuating effects on GS activity. The sites are E305 site (E305K) and site R341 (R341C), of which the latter is the congenital diseased patient site. This psGS gene was cloned from CHO-K1 cells and its GS activity was tested. It was found that the psGS protein has minimal activity compared to WT GS as shown in the bar graph on FIG. 4. Thus, FIG. 10 shows that mutation on multiple residues on the GS would attenuate the GS activity.

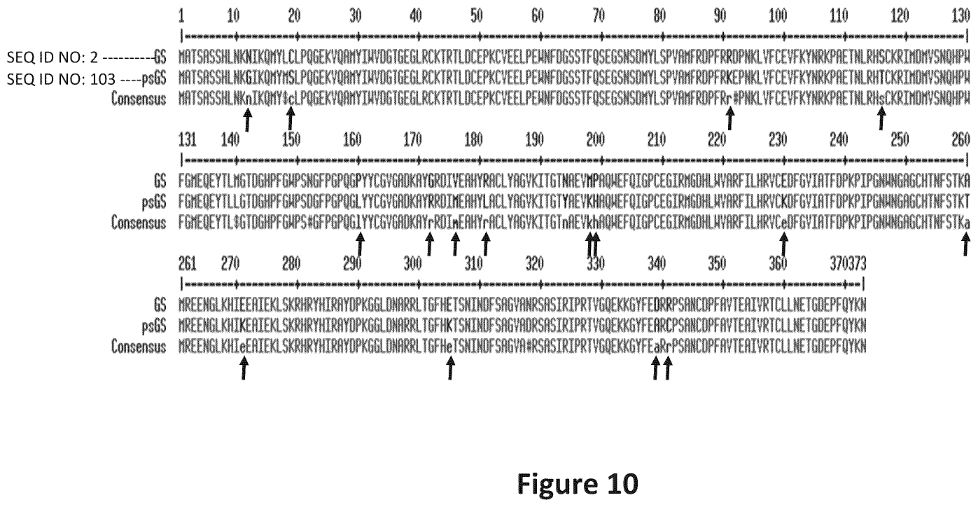

### DETAILED DESCRIPTION OF THE PRESENT INVENTION

In certain mammalian cells, particularly the Chinese hamster ovary (CHO) cells, the glutamine synthetase (GS) has been inactivated. As such, these cells cannot synthesize glutamine on their own and can only survive and grow if glutamine is added into the culture medium. Therefore, the GS gene has been widely used in expression vectors as a positive selection marker. In the field of stable cell line development for biologic production, there is a need to select for cells that are high producers and yet stable at the same time. Typically, when using the GS selection system, this is achieved by using methionine sulphoximine (MSX) to amplify the copy number of the expression cassette in the chromosome because MSX inhibits the GS activity; only the cells with higher copy number of GS can survive MSX treatment. Currently, hundreds of single clones need to be isolated to determine their productivity and their stability over at least 60 generations is assessed upon MSX removal. This is a labor intensive and time consuming process. In view of the above problems, there is a need to provide an alternative expression vector comprising a polynucleotide encoding for an alternative glutamine synthetase.

The inventors of the present disclosure have found an alternative expression vector comprising a polynucleotide encoding for an alternative glutamine synthetase, namely the GSatt system. The present invention entails the creation of a series of mutated GS gene as selection markers. The mutated GS, carries a reduced GS activity compared to the normal GS. When the attenuated GS (GSatt) is used as the selection marker for producing recombinant biologics in stably transfected cells, only the cells with the expression vector inserted into highly transcriptionally active site or the cells having several copies of the expression vectors inserted into less active site to compensate for the loss of GS activity can survive the selection. Often, the productivity of the recombinant biologics in these cells is higher because the nucleotide sequences encoding the recombinant biologics are also located in a highly transcriptionally active site or because there are multiple copies of nucleotide sequences encoding the recombinant biologics. Therefore, attenuated selection markers help to increase the selection stringency and increase the chance to identify high producers in a relatively small number stably transfected cells. Additionally, by using the GSatt system, the need to use glutamine synthetase inhibitor such as MSX can be eliminated and the stability can be enhanced as the GSatt marker provides a consistent selection pressure thereby saving effort and time in the generation of stable cell lines. Thus, in one aspect, the present invention provides an expression vector comprising a polynucleotide encoding for a glutamine synthetase with reduced activity compared to a wild type glutamine synthetase. As used herein, an expression vector generally refers to a plasmid or virus designed for gene expression in cells. The vector is used to introduce a specific gene into a target cell, and can commandeer the cell's mechanism for polypeptide synthesis to produce the polypeptide encoded by the gene. As used herein, the term “reduced activity” refers to the decrease in the ability of a mutated enzyme (such as glutamine synthetase) in synthesizing a product (such as glutamine) when compared to wild type enzyme. The activity of an enzyme having reduced activity is about 90%, or about 80%, or about 70%, or about 60%, or about 50%, or about 40%, or about 30%, or about 20%, or about 10%, or about 9%, or about 8%, or about 7%, or about 6%, or about 5%, or about 4%, or about 3%, or about 2%, or about 1.9%, or about 1.8%, or about 1.7%, or about 1.6%, or about 1.5%, or about 1.4%, or about 1.3%, or about 1.2%, or about 1.1%, or about 1%, or about 0.9%, or about 0.8%, or about 0.7%, or about 0.6%, or about 0.5%, or about 0.4%, or about 0.3%, or about 0.2%, or about 0.1% of the activity of a wild type enzyme. In one example, the activity of an enzyme having reduced activity is about 1.6%, of the activity of a wild type enzyme. In other words, if the activity of a wild type enzyme is considered as 100%, the activity of the mutated enzyme (or the enzyme having reduced activity) is reduced by about 10%, or about 20%, or about 30%, or about 40%, or about 50%, or about 60%, or about 70%, or about 80%, or about 90%, or about 91%, or about 92%, or about 93%, or about 94%, or about 95%, or about 96%, or about 97%, or about 98%, or about 98.1%, or about 98.2%, or about 98.3%, or about 98.4%, or about 98.5%, or about 98.6%, or about 98.7%, or about 98.8%, or about 98.9%, or about 99%, or about 99.1%, or about 99.2%, or about 99.3%, or about 99.4%, or about 99.5%, or about 99.6%, or about 99.7%, or about 99.8%, or about 99.9% when compared to the activity of a wild type enzyme. In one example, the activity of the mutated enzyme (or the enzyme having reduced activity) is reduced by about 98.4% when compared to the activity of a wild type enzyme. In one example, the average reduction of the mutated glutamine synthetase such as R324C mutant, D63A mutant, and E305 mutant is about 98.4% when compared to the activity of a wild type glutamine synthetase. In one example, the glutamine synthetase with reduced activity compared to a wild type glutamine synthetase or the glutamine synthetase expressed using the expression vector described herein is a recombinant glutamine synthetase. As used herein, the term “recombinant glutamine synthetase” refers to a polypeptide (such as glutamine synthetase) that results from the expression of recombinant DNA (such as expression vector described herein) within living cells (such as host cells).

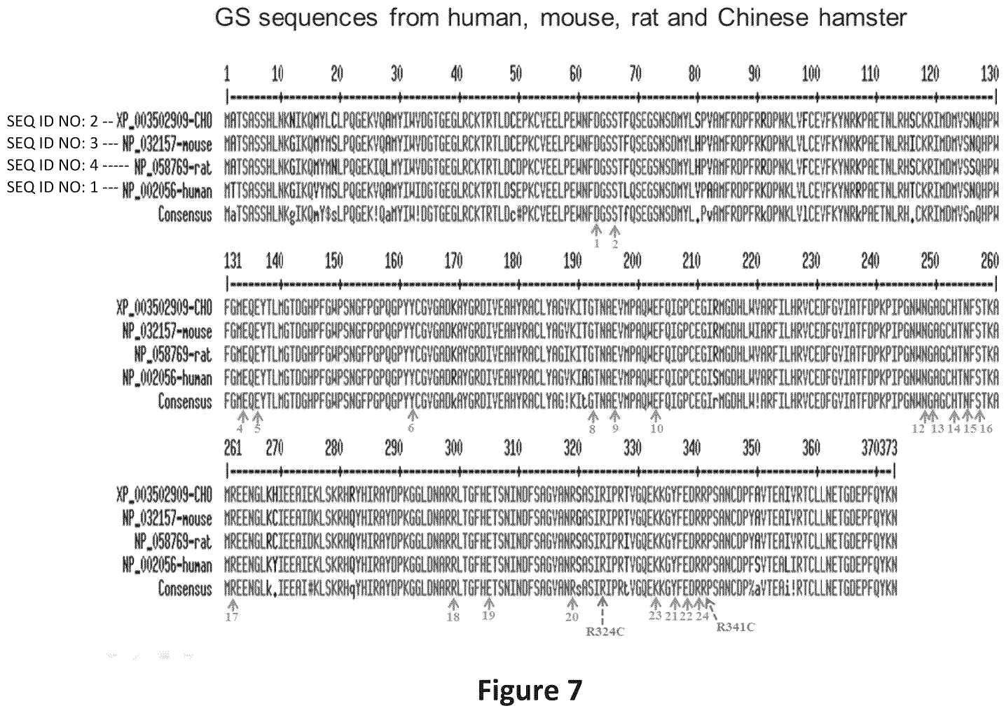

In one example, the glutamine synthetase with reduced activity encoded by the expression vector described herein comprises one or more mutations as compared to a wild type glutamine synthetase. As used herein, the term “wild type glutamine synthetase” refers to a typical form of glutamine synthetase that occurs in nature. A wild type glutamine synthetase typically comprises of two major domains, namely the beta grasp domain (from amino acid residue at position 30 to amino acid residue at position 104 in a wild type glutamine synthetase) and the catalytic domain (from amino acid residue at position 134 to amino acid residue at position 351 in a wild type glutamine synthetase). As shown for example on FIG. 7, some of the amino acid residues that are involved in ATP, glutamate or ammonia binding are located within either the beta grasp domain or the catalytic domain. Amino acid sequence of wild type glutamine synthetase sequences from are listed as SEQ ID NO: 1 (wild type human GS sequence), SEQ ID NO: 2 (wild type Chinese hamster GS sequence), SEQ ID NO: 3 (wild type mouse GS sequence), and SEQ ID NO: 4 (wild type rat GS sequence). As used herein, the term “mutation” refers to a nucleotide sequence change in an isolated nucleic acid. The nucleotide sequence change includes, but is not limited to, a missense mutation, a nonsense mutation, a nucleotide substitution, a nucleotide deletion, a nucleotide insertion, nucleotide duplication, a frameshift mutation, a repeat expansion, and the like. However, the mutation cannot include a mutation that completely inactivates the activity of an enzyme (such as glutamine synthetase). An isolated nucleic acid that bears a mutation has a nucleic acid sequence that is statistically different in sequence from a homologous nucleic acid isolated from a corresponding wild-type population. In one example, the mutation may be a substitution or a deletion. A person skilled in the art appreciate that in order to express a GS comprising at least one mutation or a mutated GS, the sequences of the polynucleotide that are used to express the wild type GS has to be altered. Any method known in the art to alter the sequences of the polynucleotide can be used. In one example, the mutation is introduced via site directed mutagenesis. Site directed mutagenesis is performed by determining the target to be mutated and by using primers suitable for introducing the mutation (as shown for example in Table 2).

In order to be able to obtain a glutamine synthetase with reduced activity compared to a wild type glutamine synthetase, the locations of the mutations have to be determined. Thus, in one example, the glutamine synthetase with reduced activity comprises one or more mutations in the glutamate binding region, the ATP binding region and/or the ammonia binding region. A person skilled in the art appreciate that protein such as glutamine synthetase comprises multiple amino acid residues that form a polypeptide chain (also known as primary structure of a protein). Due to interactions between amino acid residues in the polypeptide chain, the polypeptide chain folds and forms three-dimensional structure (also known as tertiary structure of a protein). Therefore, the term “binding region” as used herein does not necessarily refer to a series of amino acid residues that are consecutively linked (or that are located next to each other) in a polypeptide chain. That is, amino acid residues that are part of a certain binding region can be located away from each other within the polypeptide chain. However, those amino acid residues may actually be located near to each other in the three-dimensional structure of the protein and thereby forming a binding region. For example, amino acid residues that are part of the ammonia binding region are located at positions 63, 66, 162, and 305 of the polypeptide chain of a glutamine synthetase, amino acid residues that are part of the glutamate binding region are located at positions 134, 136, 196, 203, 248, 249, 253, 299, 319, 338, and 340 of the polypeptide chain of a glutamine synthetase, and amino acid residues that are part of the ATP binding region are located at positions 192, 255, 257, 262, 324, 333, and 336 of the polypeptide chain of a glutamine synthetase.

As illustrated for example in Table 1, in one example, wherein when the mutation is in the glutamate binding region, the mutation of one, or at least one, or at least two, or at least three, or at least four, or at least five, or at least six, or at least seven, or at least eight, or at least nine, or at least 10, or all of the amino acid(s) is made at a position that includes, but are not limited to, position 134, 136, 196, 203, 248, 249, 253, 299, 319, 338, 340, and the like of a glutamine synthetase, wherein the glutamine synthetase has the amino acid sequence shown in SEQ ID NO: 1, SEQ ID NO: 2, SEQ ID NO: 3 or SEQ ID NO:4. In one example, wherein when the mutation is in the glutamate binding region, the mutation of one amino acid is made at a position that includes, but are not limited to, position 134, 136, 196, 203, 248, 249, 253, 299, 319, 338, 340, and the like of a glutamine synthetase, wherein the glutamine synthetase has the amino acid sequence shown in SEQ ID NO: 1, SEQ ID NO: 2, SEQ ID NO: 3 or SEQ ID NO:4. As illustrated for example in Table 1, in one example, wherein when the mutation is in the ammonia binding region, the mutation of one, or at least one, or at least two, or at least three, or all of the amino acid(s) is made at a position that includes, but are not limited to, position 63, 66, 162, 305, and the like of a glutamine synthetase, wherein the glutamine synthetase has the amino acid sequence shown in SEQ ID NO: 1, SEQ ID NO: 2, SEQ ID NO: 3 or SEQ ID NO:4. In one example, wherein when the mutation is in the ammonia binding region, the mutation of one amino acid is made at a position that includes, but are not limited to, position 63, 66, 162, 305, and the like of a glutamine synthetase, wherein the glutamine synthetase has the amino acid sequence shown in SEQ ID NO: 1, SEQ ID NO: 2, SEQ ID NO: 3 or SEQ ID NO:4. As illustrated for example in Table 1, in one example, wherein when the mutation is in the ATP binding region, the mutation of one, or at least one, or at least two, or at least three, or at least four, or at least five, or all of the amino acid(s) is made at a position that includes, but are not limited to, position 192, 255, 257, 262, 333, 336, 324 and the like of a glutamine synthetase, wherein the glutamine synthetase has the amino acid sequence shown in SEQ ID NO: 1, SEQ ID NO: 2, SEQ ID NO: 3 or SEQ ID NO:4. In one example, wherein when the mutation is in the ATP binding region, the mutation of one amino acid is made at a position that includes, but are not limited to, position 192, 255, 257, 262, 336, 324 and the like of a glutamine synthetase, wherein the glutamine synthetase has the amino acid sequence shown in SEQ ID NO: 1, SEQ ID NO: 2, SEQ ID NO: 3 or SEQ ID NO:4. In one example, the mutation is of at least one amino acid.

As illustrated for example in FIG. 7, the mutation of at least one amino acid is made at a position wherein there is a consensus among the wild type GS sequences from Chinese hamster (SEQ ID NO: 2), mouse (SEQ ID NO: 3), rat (SEQ ID NO: 4), and human (SEQ ID NO: 1). Thus, in one example, the mutation of one, or at least one, or at least two, or at least three, or at least four, or at least five, or at least six, or at least seven, or at least eight, or at least nine, or at least 10, or at least 11, or at least 12, or at least 13, or at least 14, or at least 15, or at least 16, or at least 17, or at least 18, or at least 19, or at least 20, or at least 21, or at least 22, or all of the amino acid(s) is made at a position that includes, but are not limited to, position 12, 19, 63, 66, 91, 116, 134, 136, 160, 162, 172, 176, 181, 192, 194, 196, 198, 199, 203, 230, 248, 249, 253, 255, 257, 260, 262, 271, 299, 305, 319, 336, 338, 333, 340, 324, 341, and the like of a glutamine synthetase, wherein the glutamine synthetase has the amino acid sequence shown in SEQ ID NO: 1, SEQ ID NO: 2, SEQ ID NO: 3 or SEQ ID NO:4. In one example, the mutation of one amino acid is made at a position that includes, but are not limited to, position 12, 19, 63, 66, 91, 116, 134, 136, 160, 162, 172, 176, 181, 192, 194, 196, 198, 199, 203, 230, 248, 249, 253, 255, 257, 260, 262, 271, 299, 305, 319, 336, 338, 333, 340, 324, 341, and the like of a glutamine synthetase, wherein the glutamine synthetase has the amino acid sequence shown in SEQ ID NO: 1, SEQ ID NO: 2, SEQ ID NO: 3 or SEQ ID NO:4. Without wishing to be bound by theory, the inventors of the present invention have specifically chosen to introduce one or more mutation at the positions listed above because the amino acid residues at those positions are conserved. Additionally, they are also involved in the binding of ammonia (such as amino acid residues at positions 63, 66, 162, and 305 of a glutamine synthetase), glutamate (such as amino acid residues at positions 134, 136, 196, 203, 248, 249, 253, 299, 319, 338, and 340 of a glutamine synthetase), or ATP (such as amino acid residues at positions 192, 255, 257, 262, 324, and 336 of a glutamine synthetase). The inventors have surprisingly found that amino acid residues that are conserved and that are involved in ammonia, glutamate, or ATP binding may be critical for GS activity.

Furthermore, the inventors have also surprisingly found that the amino acid positions listed herein are crucial to the activity of the GS such that substitution of these important sites would attenuate the GS activity. In one example, the mutation in the glutamine synthetase includes, but is not limited to, R324C, R341C, D63A, S66A, E134A, E136A, Y162A, G192A, E196A, E203A, N248A, G249A, H253A, N255A, S257A, R262A, R299A, E305A, R319A, R324A, Y336A, E338A, K333A, R340A, and R341A of a glutamine synthetase, wherein the glutamine synthetase has the amino acid sequence shown in SEQ ID NO: 1, SEQ ID NO: 2, SEQ ID NO: 3 or SEQ ID NO:4. In one example, to reduce its activity, the glutamine synthetase is mutated by substituting or replacing certain amino acids using alanine (A). Without wishing to be bound by theory, alanine (A) is chosen for the substitution or replacement of certain amino acid residues because alanine (A) eliminates the side chain of an amino acid residue beyond the beta carbon and it does not exerts extreme electrostatic and/or steric effect. Thus, a person skilled in the art appreciates that the substitution or replacement of certain amino acid residues in order to reduce glutamine synthetase activity can be performed using any amino acid amino acid residue that does not have extreme electrostatic and/or steric effect. In one example, the mutation in the glutamine synthetase wherein a certain amino acid is replaced by alanine (A) includes, but is not limited to, D63A, S66A, E134A, E136A, Y162A, G192A, E196A, E203A, N248A, G249A, H253A, N255A, S257A, R262A, R299A, E305A, R319A, R324A, Y336A, E338A, K333A, R340A, and R341A of a glutamine synthetase, wherein the glutamine synthetase has the amino acid sequence shown in SEQ ID NO: 1, SEQ ID NO: 2, SEQ ID NO: 3 or SEQ ID NO:4.

In order for the expression vector described herein to be able to express the glutamine synthetase with reduced activity, a promoter is required. As used herein, the term “promoter” refers to polynucleotide sequences (such as DNA sequences) that define where transcription of a gene by RNA polymerase begins. Promoter sequences are typically located directly upstream or at the 5′ end of the transcription initiation site. Thus, in one example, in the expression vector as described herein, the polynucleotide encoding for a glutamine synthetase with reduced activity is operatively linked to a promoter. In one example, the promoter is at the 5′ direction of the polynucleotide encoding for a glutamine synthetase with reduced activity. As used herein, the term “operatively linked” is intended to mean that the two polynucleotides are connected in a manner such that each polynucleotide can serve its intended function. In one example, two polynucleotides that are operatively linked are connected by a polynucleotide linker having length of up to 10 bp, or up to 20 bp, or up to 30 bp, or up to 40 bp, or up to 50 bp, or up to 60 bp, or up to 70 bp, or up to 80 bp, or up to 90 bp, or up to 100 bp, or up to 110 bp, or up to 120 bp, or up to 130 bp, or up to 140 bp, or up to 150 bp. In one example, both polynucleotides can be transcribed and the product of the transcription can then be translated into polynucleotide.

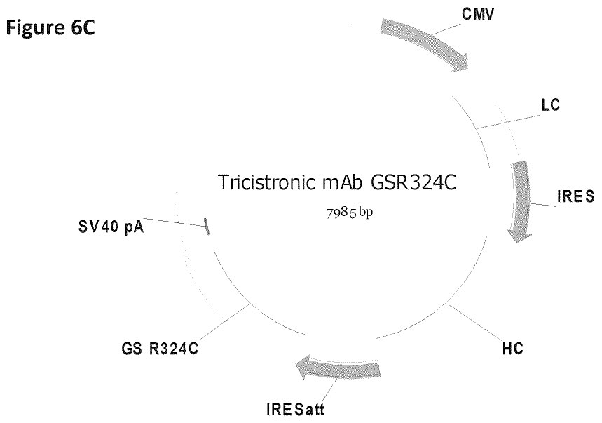

As described above, the expression vector described herein can be used for generating a stable cell lines that can produce a polypeptide of interest. In order for the polypeptide of interest to be expressed, a promoter is also required. Thus, in one example, each of the at least one polynucleotide encoding for a polypeptide of interest is operatively linked to a promoter. In one example, the promoter is at the 5′ direction of each of the at least one polynucleotide encoding for a polypeptide of interest. A person skilled in the art is aware that more than one promoter may be needed for the expression of more than one polypeptide. Thus, in one example and as shown for example in FIG. 6A, in the expression vector described herein, the promoter linked to the polynucleotide encoding for a glutamine synthetase with reduced activity is different from the promoter linked to the at least one polynucleotide encoding for a polypeptide of interest.

Further to the above and as shown for example on FIGS. 6B and 6C, in the expression vector described herein, only one of the at least one polynucleotide encoding for a polypeptide of interest is operatively linked to a promoter. In one example, the promoter is at the 5′ direction of the one polynucleotide encoding for a polypeptide of interest that is operatively linked to a promoter. In one example, the polynucleotide encoding for a glutamine synthetase with reduced activity compared to a wild type glutamine synthetase is not linked to a promoter.

As discussed above, stable cell lines generated using the expression vector described herein can be used to express polypeptide of interest. Thus, in one example, the expression vector described herein further comprising at least one polynucleotide encoding for a polypeptide of interest such as a recombinant gene product. As used herein, the term “polypeptide of interest” refers to a polypeptide that are not native to the host cell and that can be expressed at a suitable amount in a host cell using the expression vector described herein. Thus, non-limiting example of the polypeptide of interest that is encoded by the expression vector includes but is not limited to a recombinant protein and a part thereof, a fusion protein and a part thereof, an antibody and a part thereof, and the like. Any type of antibody that is made of polypeptide can be expressed using the expression vector described herein. In one example, the antibody is a monoclonal antibody. In one example, the monoclonal antibody that is encoded by the expression vector is antibody GA101 (also referred as “Antibody A” in the present disclosure). In one example, the monoclonal antibody that is encoded by the expression vector is Rituximab (also referred as “Antibody B” in the present disclosure). A person skilled in the art is aware that an antibody typically comprises light chains and heavy chains. As used herein, the term “light chains” refer to the shorter or smaller polypeptide subunit of an antibody or an immunoglobulin whereas the term “heavy chains” refer to the longer or larger polypeptide subunit of an antibody or an immunoglobulin. Thus, in one example, the polypeptide of interest that is encoded by the expression vector described herein is the heavy chain of an antibody of interest. In one example, the polypeptide of interest that is encoded by the expression vector described herein is the light chain of an antibody of interest. The heavy chain and the light chain of the antibody of interest as described herein can both be expressed by one expression vector. Thus, in one example, the expression vector as described herein comprises two polynucleotides each encoding for a polypeptide of interest, wherein one of the polynucleotides encodes for the light chain of an antibody of interest and one of the polynucleotides encodes for the heavy chain of the same antibody of interest.

As shown for example in FIGS. 6A-6C, in the expression vector as described herein, the polynucleotide encoding for the light chain of an antibody of interest and the polynucleotide encoding for the heavy chain of the same antibody of interest are separated by an internal ribosome entry site. As used herein, the term “internal ribosome entry site” or “IRES” refers to polynucleotide sequences in an expression vector (such as a multicistronic, bicistronic, or tricistronic expression vector), which when transcribed into mRNA, are believed to recruit ribosomes directly, without a previous scanning of untranslated region of mRNA by the ribosomes. IRESs are commonly used to direct the expression of the second cistrons of bicistronic mRNAs. As used herein, the term “multicistronic expression vector” refers to an expression vector that comprises of multiple cistrons multiple sections that encodes for multiple polypeptides. Thus, a multicistronic expression vector can be transcribed into an mRNA that can simultaneously expresses two or more separate polypeptides. Therefore, a bicistronic expression vector is an expression vector comprising two cistrons and an mRNA transcribed from a bicistronic expression vector can simultaneously express two separate polypeptides. A tricistronic expression vector is an expression vector comprising three cistrons and an mRNA transcribed from a tricistronic expression vector can simultaneously express three separate polypeptides.

The inventors have found that the ability of the expression vector described herein to express the light chain and the heavy chain of an antibody of interest is not negatively affected by the position of the polynucleotides encoding for the light chain and for the heavy chain of an antibody of interest. Having said that, it is generally understood that the expression level of the polynucleotides in an IRES containing vector is affected by the position of the polynucleotides with respect to the IRES. The first polypeptide (i.e. the polypeptide that is encoded by polynucleotide located before the IRES) will have a higher expression level than the second polypeptide that is encoded by the polynucleotide located after the IRES. Thus, in one example, in the expression vector as described herein, the polynucleotide encoding for the light chain of an antibody of interest is in the 5′ direction of the polynucleotide encoding for the heavy chain of the same antibody of interest. In another example, in the expression vector as described herein, the polynucleotide encoding for the heavy chain of an antibody of interest is in the 5′ direction of the polynucleotide encoding for the light chain of the same antibody of interest.

As also shown for example in FIGS. 6A-6C, the polynucleotide encoding the antibody of interest is located upstream or is in the 5′ direction of the polynucleotide encoding for a glutamine synthetase with reduced activity. Those polynucleotides are separated by an internal ribosome entry site (IRES). Thus, in one example, in the expression vector as described herein, the polynucleotide encoding for the heavy chain of an antibody of interest is separated from the polynucleotide encoding for a glutamine synthetase with reduced activity by an internal ribosome entry site. In another example, in the expression vector as described herein, the polynucleotide encoding for the light chain of an antibody of interest is separated from the polynucleotide encoding for a glutamine synthetase with reduced activity by an internal ribosome entry site. As used herein, the term “separated from” means that the locations of polynucleotides encoding for a polypeptide that are not located right next to another polynucleotides encoding for another polypeptides. In between those two polynucleotides that encode two different polypeptides, there are polynucleotides encoding other sequences (such as encoding internal ribosome entry site or IRES).

Further to the above, as also shown for example in FIGS. 6B and 6C, an expression vector as described herein may comprise more than one internal ribosome entry sites (IRES). Thus, in one example, the internal ribosome entry site separating the polynucleotide encoding for the light chain of an antibody of interest and the polynucleotide encoding for the heavy chain of the same antibody of interest is different from the internal ribosome entry site separating the polynucleotide encoding for the light/heavy chain of an antibody of interest from the polynucleotide encoding for a glutamine synthetase with reduced activity. When an expression vector as described herein comprises more than one internal ribosome entry sites (IRES), the internal ribosome entry sites can be of multiple types and can have different translation efficiencies. Thus, in one example, in the expression vector described herein, the internal ribosome entry site separating the polynucleotide encoding for the light chain of an antibody of interest and the polynucleotide encoding for the heavy chain of the same antibody of interest is a wild type internal ribosome entry site, and wherein the internal ribosome entry site separating the polynucleotide encoding for the light/heavy chain of an antibody of interest from the polynucleotide encoding for a glutamine synthetase with reduced activity is an internal ribosome entry site with attenuated translation efficiency. As shown for example in FIGS. 6B and 6C, the wild type internal ribosome entry site is denoted as “IRES” and the internal ribosome entry site with attenuated translation efficiency is denoted as “IRESatt”. As used herein, the term “internal ribosome entry site with attenuated translation efficiency” or “IRESatt” refers to an IRES wherein its 3′ region has been modified such that its translation efficiency is reduced when compared to a wild type IRES.

Further to the above, a person skilled in the art is aware that the 3′ ends of most mammalian mRNAs are polyadenylated or are connected with multiple adenine that forms poly(A) tail. Poly(A) tail is essential for the survival, transport, stability, and translation of most mRNAs. In order to provide for the poly(A) tail, the expression vector has to comprise one or more polyadenylation signals. When more than one polyadenylation signals are provided, the polyadenylation signal can be located at the 3′ end of each set of polynucleotide sequences that encode for a polypeptide. Thus, in one example, the expression vector as described herein comprises one polyadenylation signal at the 3′ end of each of the at least one polynucleotide encoding for a polypeptide of interest, and one polyadenylation signal at the 3′ end of the polynucleotide encoding for a glutamine synthetase with reduced activity. As shown for example on FIG. 6A, the expression vector comprises two polyadenylation signals, wherein one signal is located at the 3′ end of the polynucleotide encoding the heavy chain of an antibody of interest and wherein the other signal is located at the 3′end of the polynucleotide encoding for a glutamine synthetase with reduced activity. In another example, the expression vector as described herein comprises one polyadenylation signal only at the 3′ end of the polynucleotide encoding for a glutamine synthetase with reduced activity. As shown for example on FIGS. 6B and 6C, the expression vector comprises of only one polyadenylation signal, which is located at the 3′end of the polynucleotide encoding for a glutamine synthetase with reduced activity.

As discussed above, to generate stable cell lines using glutamine synthetase with reduced activity as a selection marker, the expression vector as described herein is inserted or incorporated into a host cell. Thus, in one aspect, the present invention provides a host cell comprising the expression vector as described herein. Prior to the insertion or the incorporation of the expression vector described herein, the host cell does not have any glutamine synthetase activity or the host cell has minimum amount of glutamine synthetase activity. As used herein, the term “minimum amount of glutamine synthetase activity” refers to an amount of glutamine synthetase activity that does not allow the host cells to grow and/or survive in the absence of L-glutamine supplementation. After the insertion or the incorporation of the expression vector described herein into the host cell, glutamine synthetase having reduced activity is produced. Therefore, the host cell comprising the expression vector described herein will have glutamine synthetase activity but the glutamine synthetase activity of said host cells will be lower than the glutamine synthetase activity of a wild type cell.

A person skilled in the art appreciated that any type of host cell that does not have glutamine synthetase activity prior to the insertion or incorporation of the expression vector described herein can be used. In one example, the host cells can be prepared from any cells (such as mammalians or yeast cells) that typically have genes that encode for wild type or mutant or attenuated glutamine synthetase and wherein said gene has been knocked out. As used herein, the term “knocked out” refers to genes that have been inactivated and hence cannot express protein of interest. Cells wherein the glutamine synthetase encoding genes have been knocked out lose their glutamine synthetase activity and cannot survive in glutamine-free environment without insertion or incorporation of the expression vector described herein. Therefore, said cells are suitable as host cells for the present disclosure. In one example, the host cell comprising the expression vector described herein is a mammalian cell. Non limiting example of mammalian cell that can be used as a host cell of the expression vector described herein includes, but is not limited to, Chinese hamster ovary (CHO) cell line, human embryonic kidney 293 (HEK293) cell line, cell lines derived from mouse, cell lines derived from rat, cell lines derived from human, and the like. In one example, the host cell comprising the expression vector described herein is a Chinese hamster ovary (CHO) cell line. In one example, the host cell comprising the expression vector described herein is a CHOK1 GFT^{-/-} GS^{-/-} cell line or a CHOK1 GS^{-/-}. As used herein the term “GFT” stands for GDP fucose transporter. As used herein, the symbol “−/−” refers to the fact that the cell is deficient of certain enzyme. For example, GFT^{-/-} denotes that the cell is deficient of GDP fucose transporter (GFT) and GS^{-/-} denotes that the cell is deficient of glutamine synthetase (GS). In one example, the host cell comprising the expression vector described herein is a yeast cell. Non limiting example of yeast cell that can be used as a host cell of the expression vector described herein includes, but is not limited to, cell lines derived from Saccharomyces cerevisiae, and the like.

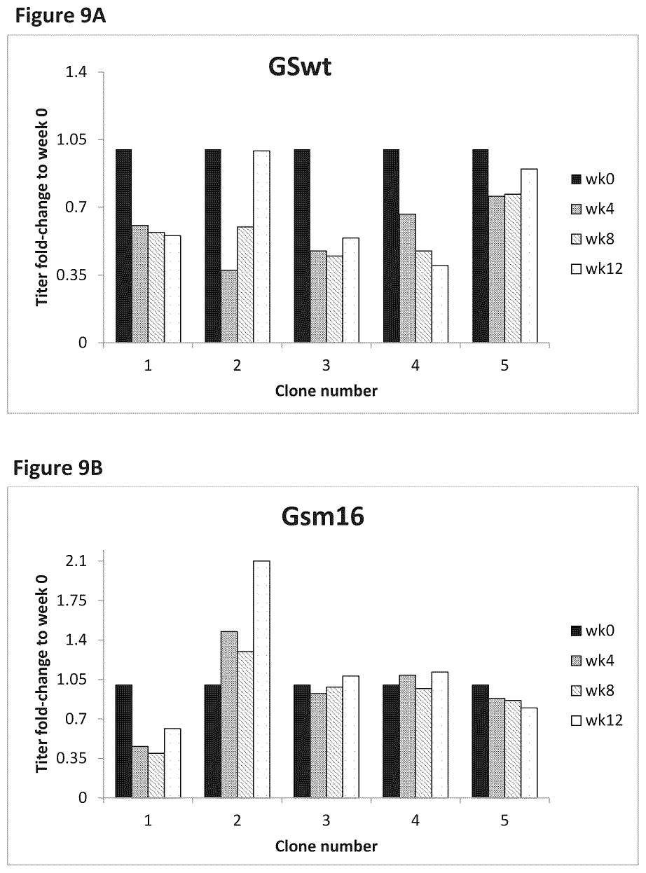

In addition to providing an alternative expression vector and a host cell comprising said expression vector, as shown for example in FIGS. 9A-9B, the inventors of the present invention have also provided an alternative method for preparing stable cell line. Thus, in yet another aspect, the present invention provides a method for preparing stable cell line, comprising: (a) transforming a host cell having minimum amount of glutamine synthetase activity or no glutamine synthetase activity with the expression vector described herein; (b) culturing the transformed host cell in a medium that selectively allows the proliferation of the transformed host cells comprising an amplified number of copies of the vector to be selected. In yet another aspect, the present invention provides a method for preparing stable cell line, comprising: (a) transforming a host cell having no glutamine synthetase activity with the expression vector described herein; (b) culturing the transformed host cell in a medium that selectively allows the proliferation of the transformed host cells comprising an amplified number of copies of the vector to be selected. As used herein, the term “stable cell line” refers to cells comprising expression vector described herein that are able to sustainably express a polypeptide of interest.

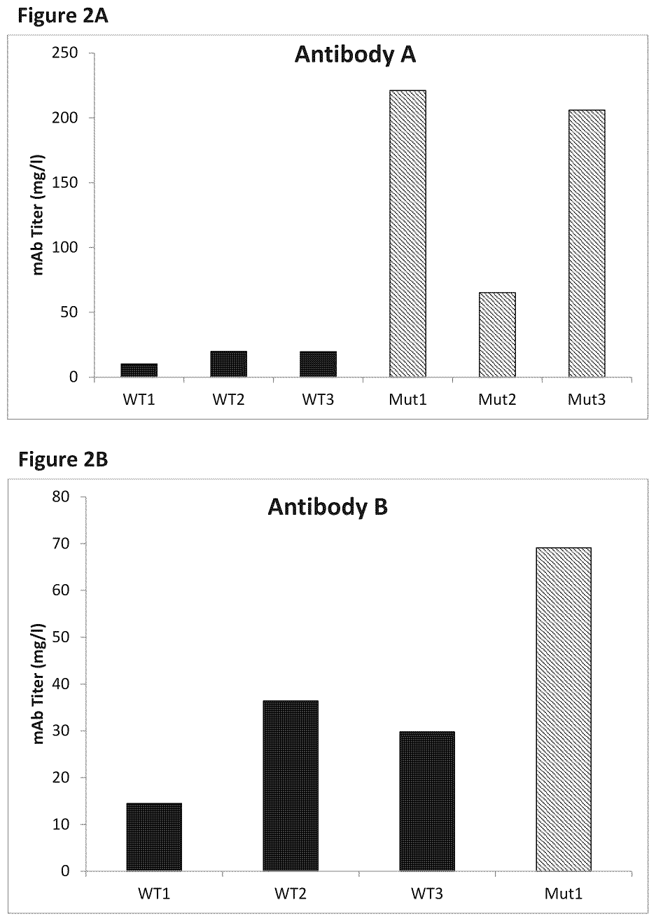

Further to the above, as shown for example in FIGS. 2A-2B, the inventors of the present invention have also provided an alternative method for producing antibody polypeptide of interest. Thus, in yet another aspect, the present invention provides a method of producing polypeptide of interest, comprising: (a) transforming a host cell having minimum amount of glutamine synthetase activity or no glutamine synthetase activity with the expression vector described herein; (b) culturing the transformed host cell in a medium that selectively allows the production of polypeptide of interest; and (c) collecting the polypeptide of interest from the medium of (b). In yet another aspect, the present invention provides a method of producing polypeptide of interest, comprising: (a) transforming a host cell having no glutamine synthetase activity with the expression vector described herein; (b) culturing the transformed host cell in a medium that selectively allows the production of polypeptide of interest; and (c) collecting the polypeptide of interest from the medium of (b). Collection of the polypeptide of interest from the cell culture medium can be performed using any method known in the art. Non limiting examples of method to collect the polypeptide of interest from the medium include, but are not limited to, size exclusion chromatography, hydrophobic interaction chromatography, ion exchange chromatography, free-flow-electrophoresis, affinity chromatography, immunoaffinity chromatography, high performance liquid chromatography, and the like.

As used herein, the term “transformation” refers to the genetic alternation of a cell resulting from the direct uptake and incorporation of exogenous genetic materials from its surrounding. The term “transforming a host cell” thus refers to the act of introducing or contacting exogenous genetic material such as the expression vector described herein to the host cell. The term “transformed host cell” thus refers to a host cell into which an exogenous genetic material such as the expression vector described herein has been inserted or incorporated. Transformation of a host cells with the expression vector described herein can be performed using any method known in the art. In one example of the method described herein, the medium that allow the transformed host cells comprising an amplified number of copies of the vector to be selected is a glutamine free cell culture medium. In one example of the method described herein, the transformed host cells are cultured under glutamine free conditions. Without wishing to be bound by theory, when the host cells are cultured under glutamine free conditions, host cells that are not inserted by the expression vector described herein or the host cells that do not incorporate the expression vector described herein will not be able to survive and/or proliferate under the glutamine free condition. The inventors have also surprisingly found that when the attenuated GS (GSatt) or a glutamine synthetase with reduced activity compared to a wild type glutamine synthetase is used as the selection marker for producing antibody polypeptide of interest in stably transfected cells, only the cells with the expression vector inserted into highly transcriptionally active site or the cells having several copies of the expression vectors inserted into less active site to compensate for the loss of GS activity can survive the selection. Therefore, unlike when wild type GS is used as a selection marker, the selection of cells using GS inhibitor (i.e. selection for cells having higher copy number of wild type GS) may not be required. Thus, in one example, the method described herein does not comprise the use of a glutamine synthetase inhibitor. The glutamine synthetase inhibitor that is not used in the method described herein can include any glutamine synthetase inhibitor known in the art. In one example, the glutamine synthetase inhibitor that is not used in the method described herein is methionine sulphoximine (MSX).

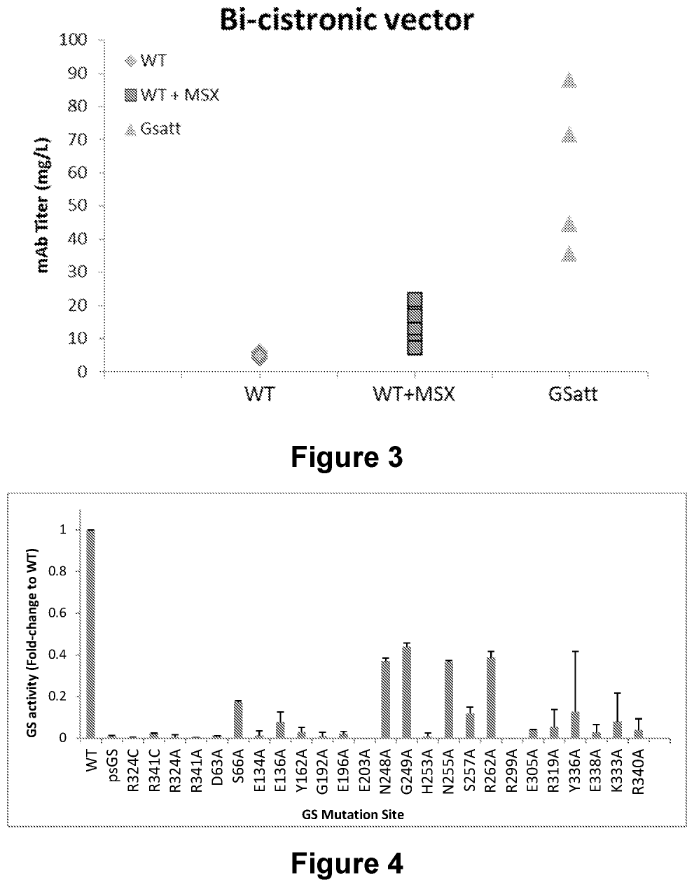

The inventors have surprisingly found that the use of attenuated GS as a selection marker provides an advantage over the use of wild type GS (WT GS) as a selection marker. As shown for example in FIG. 3, the amount of mAb titer in the batch culture stable pools transfected with the mutant GS are generally higher than those WT GS pools treated with MSX (25 μM). The average fold-enhancement is about 9-fold. This is higher than the 2 fold-enhancement between WT GS and WT GS plus MSX that is observed in the prior art.

More importantly, the use of attenuated GS as a selection marker improves production stability. Attenuation of other selection markers like neomycin phosphotransferase II and Dihydrofolate reductase (DHFR) genes through mutation have been previously developed for enhancing the production titer. However, attenuation of those other selection markers still require the use of drug to enhance the productivity. The use of drug induces a problem in the stable cell line generation process where stability testing upon drug removal is required. Stability in production is often tested by isolating hundreds of clones and measuring their production over 60 generations (2 to 3 months). This is a laborious and time consuming process. On the other hand, the use of expression vector described herein (i.e. an expression vector comprising polynucleotide encoding attenuated mutant GS) mimics the inhibition of GS activity via GS inhibitor such as MSX drug. As the mutant GS constantly acts as a selection pressure, the production stability would be enhanced. Hence, the mutant GS creates an advantage in the stability testing.

In yet another aspect, the present invention provides a kit comprising the expression vector described herein. In one example, the kit comprising the expression vector described herein further comprises at least one of the following: (i) a host cell having no glutamine synthetase activity compared to a wild type cell; and (ii) a transfection medium or means to carry out a transfection. In one example, the kit as described herein further comprises a cell culture medium. In one example, the cell culture medium does not contain glutamine. In one example, the kit as described herein further comprises a glutamine free cell culture medium.

As used in this application, the singular form “a,” “an,” and “the” include plural references unless the context clearly dictates otherwise. For example, the term “an expression vector” includes a plurality of expression vectors, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of certain stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Material and Methods

Generation of Tricistronic mAb GS Vector.

The GS gene was amplified from the CHO cells cDNA library. The DHFR gene of the tricistronic DHFR vector was replaced with the GS gene via standard cloning methods. The primers used were:

GSsmaUP (SEQ ID NO: 31):
GTGTGACCCGGGAGATGAGGATCGTTTCGCATGGCCACCTCAGCAAGTTC
CCACTTG;
and
GSBstBILP (SEQ ID NO: 32):
GAATTCTTCGAATTAGTTTTTGTATTCGAAGGGCTCGTCGCC.

The mAb heavy and light chains were synthesized commercially and subsequently cloned into the region after the CMV promoter of the tricistronic GS vector in the following orientation: Light chain-IRES-heavy chain-IRESatt GS.

Generation of Bicistronic mAb GS Vector.

pcDNA3.1(+) from Invitrogen was used as the vector backbone. The full length heavy and light chains were cloned into the MCS of the vector as a bicistronic construct: light chain-IRES-heavy chain. The neomycin gene after the SV40 promoter was replaced with the WT GS construct. The primers used for amplifying the GS gene from the tricistronic mAb GS vector were:

GSSMASV4UP (SEQ ID NO: 33):
GTGCTCCCGGGAGCTTGTATATCCATTTTCGGATCTGATCAAGAGACAGG
ATGAGGATCGGCCACCATGGCCACCTCAGCAAGTTCCCACTTGAAC;
and
GSBSTBISV4LP (SEQ ID NO: 34):
GGTCATTTCGAACCCCAGAGTCCCGCTTAGTTTTTGTATTGGAAGGGCTC
GTCG

Generation of the GS-IRES-Luciferase Construct (GS-Luc).

The GS gene was cloned into the MCS region of the pcDNA3.1(+) vector. The IRES construct was cloned in after the GS gene and followed by the firefly luciferase gene. This expression cassette of GS-IRESLuciferase was used to evaluate the GS activity in which the luciferase activity was used to normalize the GS expression level.

Generation of the pseudoGS Construct

The coding sequence of the pseudo Glutamine Synthetase gene (psGS gene) was amplified from the genomic DNA of CHO-K1 cells. The psGS gene was then cloned into the GS-Luc construct (as discussed above) to replace the original GS gene to form the expression cassette of psGS-IRESLuciferase. The GS activity assay of this construct was performed as described below.

GS Activity Assay.

Adherent CHO-K1 GS^{-/-} cells were cultured in DMEM with 10% FBS in 37° C., 5% CO₂ incubator. The cells were transfected with the various GS/GSm-Luc constructs via LTX transfection reagent (Invitrogen). The cells were harvested 24 hrs post-transfection with the Reporter Lysis buffer (Promega) and their luciferase activity was measured according to the protocol of the Luciferase Assay kit (Promega). The GS activity was measured using the standard GS activity assay whereby GS-catalyzed formation of γ-glutamylhydroxamate from glutamine and hydroxylamine was measured at 500 nm. Briefly, the cell lysate supernatant was incubated with shaking at 37° C. for 45 mins with 100 mM Imidazole-HCl (pH 7), 50 mM L-Glutamine, 0.4 mM MnCl₂, 62.5 mM Hydroxylamine (pH 7) and 10 mM sodium arsenate to a final volume 250 μl. The reaction was terminated by the addition of 250 μl FeCl₃ reagent (0.37M FeCl₃, 0.67M HCl, 0.2M TCA). Precipitate was removed by centrifugation at 10,000 rpm for 5 mins and the absorbance of supernatant was measured at 500 nm using a spectrophotometer. The activity of the GS was normalized over that of the luciferase level.

Mutagenesis of the GS Gene.

Mutagenesis of the specific sites were achieved by designing primers with the specific mutations. The sites targeted (as shown on Table 1 below) were conserved between human and Chinese hamster GS. QuikChange site directed mutagenesis reaction of the Tricistronic mAb GS/bicistronic mAb GS/or the GSLuc vectors were performed using the primers listed in Table 2 below. Sequencing was performed to ensure that the mutations were achieved.

TABLE 1
Legend of the GS Mutation Sites

| | | Amino Acid at said position |
|---|---|---|
| Name | Mutation | typically involved in |
| R324C | Identified as congenital | ATP binding |
| | GS mutation | |
| R341C | Identified as congenital | N/A |
| | GS mutation | |
| 1 | D63A | Ammonia binding |
| 2 | S66A | Ammonia binding |
| 4 | E134A | Glutamate binding |
| 5 | E136A | Glutamate binding |
| 6 | Y162A | Ammonia binding |
| 8 | G192A | ATP binding |
| 9 | E196A | Glutamate binding |
| 10 | E203A | Glutamate binding |
| 12 | N248A | Glutamate binding |
| 13 | G249A | Glutamate binding |
| 14 | H253A | Glutamate binding |
| 15 | N255A | ATP binding |
| 16 | S257A | ATP binding |
| 17 | R262A | ATP binding |
| 18 | R299A | Glutamate binding |
| 19 | E305A | Ammonia binding |
| 20 | R319A | Glutamate binding |
| 21 | Y336A | ATP binding |
| 22 | E338A | Glutamate binding |
| 23 | K333A | ATP binding |
| 24 | R340A | Glutamate binding |

TABLE 2
List of primers for GS mutation

| SEQ ID NO | Name | Sequence |
|---|---|---|
| SEQ ID NO: | R324C | CGCAGTGCCAGCATCTGCATTCCCCGGACTGTCGGC |
| 35 | FP | |
| SEQ ID NO: | R324C | GCCGACAGTCCGGGGAATGCAGATGCTGGCACTGCG |
| 36 | LP | |
| SEQ ID NO: | R341C | GGTTACTTTGAAGACCGCTGCCCCTCTGCCAATTGTGAC |
| 37 | FP | |
| SEQ ID NO: | R341C | GTCACAATTGGCAGAGGGGCAGCGGTCTTCAAAGTAACC |
| 38 | LP | |
| SEQ ID NO: | R324A | CGCAGTGCCAGCATCGCCATTCCCCGGACTGTC |
| 39 | UP | |
| SEQ ID NO: | R324A LP | GACAGTCCGGGGAATGGCGATGCTGGCACTGCG |
| 40 | | |
| SEQ ID NO: | R341A | TACTTTGAAGACCGCGCCCCCTCTGCCAATTGT |
| 41 | UP | |
| SEQ ID NO: | R341A LP | ACAATTGGCAGAGGGGGCGCGGTCTTCAAAGTA |
| 42 | | |
| SEQ ID NO: | GSM1 UP | CCTGAGTGGAATTTTGCTGGCTCTAGTACCTTTCAG |
| 43 | | |
| SEQ ID NO: | GSM1 LP | CTGAAAGGTACTAGTGCCAGCAAAATTCCACTCAGG |
| 44 | | |
| SEQ ID NO: | GSM2 UP | TGGAATTTTGATGGCTCTGCTACCTTTCAGTCTGAGGGC |
| 45 | | |
| SEQ ID NO: | GSM2 LP | GCCCTCAGACTGAAAGGTAGCAGAGCCATCAAAATTCCA |
| 46 | | |
| SEQ ID NO: | GSM4 UP | CACCCCTGGTTTGGAATGGCACAGGAGTATACTCTG |
| 47 | | |
| SEQ ID NO: | GSM4 LP | CAGAGTATACTCCTGTGCCATTCCAAACCAGGGGTG |
| 48 | | |
| SEQ ID NO: | GSM5 UP | TGGTTTGGAATGGAACAGGCGTATACTCTGATGGGAACA |
| 49 | | |
| SEQ ID NO: | GSM5 LP | TGTTCCCATCAGAGTATACGCCTGTTCCATTCCAAACCA |
| 50 | | |
| SEQ ID NO: | GSM6 UP | CCCCAAGGTCCGTATGCCTGTGGTGTGGGCGCAGAC |
| 51 | | |
| SEQ ID NO: | GSM6 LP | GTCTGCGCCCACACCACAGGCATACGGACCTTGGGG |
| 52 | | |
| SEQ ID NO: | GSM8 UP | GGGGTCAAGATTACAGCAACAAATGCTGAGGTC |
| 53 | | |
| SEQ ID NO: | GSM8 LP | GACCTCAGCATTTGTTGCTGTAATCTTGACCCC |
| 54 | | |
| SEQ ID NO: | GSM9 UP | ACAGGAACAAATGCTGCGGTCATGCCTGCCCAG |
| 55 | | |
| SEQ ID NO: | GSM9 LP | CTGGGCAGGCATGACCGCAGCATTTGTTCCTGT |
| 56 | | |
| SEQ ID NO: | GSM10 | ATGCCTGCCCAGTGGGCATTCCAAATAGGACCC |
| 57 | UP | |
| SEQ ID NO: | GSM10 | GGGTCCTATTTGGAATGCCCACTGGGCAGGCAT |
| 58 | LP | |
| SEQ ID NO: | GSM12 | ATTCCTGGGAACTGGGCAGGTGCAGGCTGCCATACC |
| 59 | UP | |
| SEQ ID NO: | GSM12 | GGTATGGCAGCCTGCACCTGCCCAGTTCCCAGGAAT |
| 60 | LP | |
| SEQ ID NO: | GSM13 | CCTGGGAACTGGAATGCTGCAGGCTGCCATACC |
| 61 | UP | |
| SEQ ID NO: | GSM13 | GGTATGGCAGCCTGCAGCATTCCAGTTCCCAGG |
| 62 | LP | |
| SEQ ID NO: | GSM14 | AATGGTGCAGGCTGCGCAACCAACTTTAGCACC |
| 63 | UP | |
| SEQ ID NO: | GSM14 | GGTGCTAAAGTTGGTTGCGCAGCCTGCACCATT |
| 64 | LP | |
| SEQ ID NO: | GSM15 | GCAGGCTGCCATACCGCATTTAGCACCAAGGCC |
| 65 | UP | |
| SEQ ID NO: | GSM15 | GGCCTTGGTGCTAAATGCGGTATGGCAGCCTGC |
| 66 | LP | |
| SEQ ID NO: | GSM16 | GGCTGCCATACCAACTTTGCAACCAAGGCCATGCGG |
| 67 | UP | |
| SEQ ID NO: | GSM16 | CCGCATGGCCTTGGTTGCAAAGTTGGTATGGCAGCC |
| 68 | LP | |
| SEQ ID NO: | GSM17 | AGCACCAAGGCCATGGCGGAGGAGAATGGTCTG |
| 69 | UP | |
| SEQ ID NO: | GSM17 | CAGACCATTCTCCTCCGCCATGGCCTTGGTGCT |
| 70 | LP | |
| SEQ ID NO: | GSM18 | CTGGACAATGCCCGTGCTCTGACTGGGTTCCAC |
| 71 | UP | |
| SEQ ID NO: | GSM18 | GTGGAACCCAGTCAGAGCACGGGCATTGTCCAG |
| 72 | LP | |
| SEQ ID NO: | GSM19 | CTGACTGGGTTCCACGCAACGTCCAACATCAAC |
| 73 | UP | |
| SEQ ID NO: | GSM19 | GTTGATGTTGGACGTTGCGTGGAACCCAGTCAG |
| 74 | LP | |
| SEQ ID NO: | GSM20 | GCTGGTGTCGCCAATGCCAGTGCCAGCATCCGC |
| 75 | UP | |
| SEQ ID NO: | GSM20 | GCGGATGCTGGCACTGGCATTGGCGACACCAGC |
| 76 | LP | |
| SEQ ID NO: | GSM21 | CAGGAGAAGAAAGGTGCTTTTGAAGACCGCCGC |
| 77 | UP | |
| SEQ ID NO: | GSM21 | GCGGCGGTCTTCAAAAGCACCTTTCTTCTCCTG |
| 78 | LP | |
| SEQ ID NO: | GSM22 | AAGAAAGGTTACTTTGCAGACCGCCGCCCCTCTGCC |
| 79 | UP | |
| SEQ ID NO: | GSM22 | GGCAGAGGGGCGGCGGTCTGCAAAGTAACCTTTCTT |
| 80 | LP | |
| SEQ ID NO: | GSM23 | ACTGTCGGCCAGGAGGCGAAAGGTTACTTTGAA |
| 81 | UP | |
| SEQ ID NO: | GSM23 | TTCAAAGTAACCTTTCGCCTCCTGGCCGACAGT |
| 82 | LP | |
| SEQ ID NO: | GSM24 | GGTTACTTTGAAGACGCCCGCCCCTCTGCCAAT |
| 83 | UP | |
| SEQ ID NO: | GSM24 | ATTGGCAGAGGGGCGGGCGTCTTCAAAGTAACC |
| 84 | LP | |

Notes:
Primers designated as “R324C” and “R341C” introduce point mutation wherein Arginine (R) is mutated to Cysteine (C) at amino acid position 324 and position 341.
Primers designated as “R324A” and “R341A” introduce point mutation wherein Arginine (R) is mutated to Alanine (A) at amino acid position 324 and position 341.
Primers designated as “GSM##” introduce point mutation wherein the amino acid residue is mutated to Alanine (A). The number on “GSM##” corresponds to the column titled “Name” in Table 1. For example, primer designated as GSM1 corresponds to a primer that introduces D63A mutation (i.e. point mutation wherein Aspartic acid (D) at amino acid position 63 is mutated to Alanine (A)) and so on.
Primers that are designated as “UP” or “FP” are forward primer. Primers that are designated as “LP” are reverse primer

Cell Culture, Transfection and Stable Pool Generation.

Suspension CHO-K1 GS^{-/-} cells were cultured in HyClone PF CHO mixed with CD CHO media in 1:1 ratio (50/50) and incubated in 37° C., 8% CO₂ shaking incubator. The 50/50 media was supplemented with 0.05% Pluronic F-68 acid and 6 mM L-Glutamine. Cells were transfected with the Tricistronic/Bicistronic mAb GS/GSm constructs via Electroporation (LONZA SG kit) in the following conditions: 10 million cells with 5 ug of DNA. Two days post transfection, the transfected cells were placed under the L-glutamine free selection till their viability recovered to more than 90%. Batch culture was then performed on the recovered pools to measure the level of IgG production. IgG level was measured using the Nephelometer with the IgGC assay reagent.

Experimental Result

Transfection of CHO-K1 GFT^{-/-} GS^{-/-} with Either Tricistronic mAb-GS or Tricistronic mAb-GSatt (R324C)—Minipools Format.

Mutation of the GS gene at site −R324 to C (GSatt), corresponding to the glutamate binding region of the enzyme was made. The vector encoding an antibody gene and the GSatt selection marker in the tricistronic vector (Ho S C et. al., 2012) was compared with the wild type GS in the same format. The comparison in stable productivity level was done in CHO-K1-GFT^{-/-} GS^{-/-} cells. The stable productions of 2 different antibodies were tested.

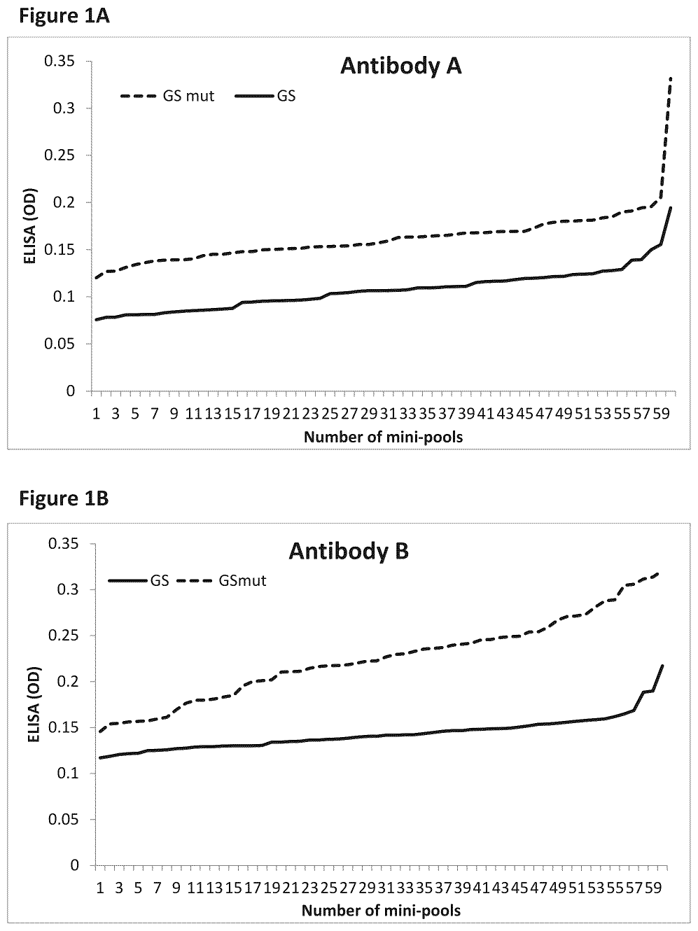

1×10⁷ cells with 5 μg of DNA per transfection was performed for each of the construct. Two different antibodies were tested—Antibody A and B. Three days post-transfection, a small aliquot from WT GS and GSatt transfectants were each seeded into a 96 well plate at 2000 cells/well in 50/50 media (CD CHO/Hyclone) without L-Glutamine selection media. The cells were passaged once 2 weeks later and incubated for another 2 weeks for overgrowth assay. The supernatants were collected and their OD readings were evaluated in an ELISA format using anti-human antibody as the capture agent. The OD readings for both WT GS and GSatt were collected and tabulated in FIGS. 1A-1B. Overall, the GSatt minipools showed an enhanced antibody production level over that of WT GS for both GA101 and Rituximab.

Transfection of CHOK1 GFT^{-/-} GS^{-/-} with Either Tricistronic mAb-GS or Tricistronic mAb-GSatt (R324C)—Stable Suspension Pool Format.

Mutation of the GS gene at site −R324 to C (GSatt), corresponding to the glutamate binding region of the enzyme was made. The vector encoding an antibody gene and the GSatt selection marker in the tricistronic vector (Ho S C et. al., 2012) was compared with the wild type GS in the same format. The comparison in stable productivity level was done in CHO-K1 GFT^{-/-} GS^{-/-} cells. The stable productions of 2 different antibodies were tested.

Three transfections per construct (1×10⁷ cells with 5 μg of DNA per transfection) were performed. Two different antibodies were tested—Antibody A and B. The transfectant pools were selected in 50/50 media (CD CHO/Hyclone) without L-Glutamine until they recover. Upon recovery, a batch culture was performed to determine the productivity. The WT GS and GSatt pools took an average of about 3.5 weeks and 6.5 weeks to recover respectively before the batch culture was performed.

The experimental result for Antibody A was shown in FIG. 2A. All 3 pools with the GSatt selection maker (Mut1-3) demonstrated significantly higher level of mAb titer over that of the wild type GS (WT1-3). The GSatt pools showed at average of 9.9-fold more titer than the WT GS pools. The experimental result for Antibody B was shown in FIG. 2B. Two pools for the Antibody B-GSatt did not survive. The remaining pool (Mut1) showed at least 2.6× more titer than the average of the 3 WT pools. Based on the results shown on FIGS. 2A and 2B, the use of GSatt can enhance the stable production of 2 different antibodies in CHO cells.

Several Novel Sites on the GS, when Mutated to Alanine, would Attenuate the GS Activity.

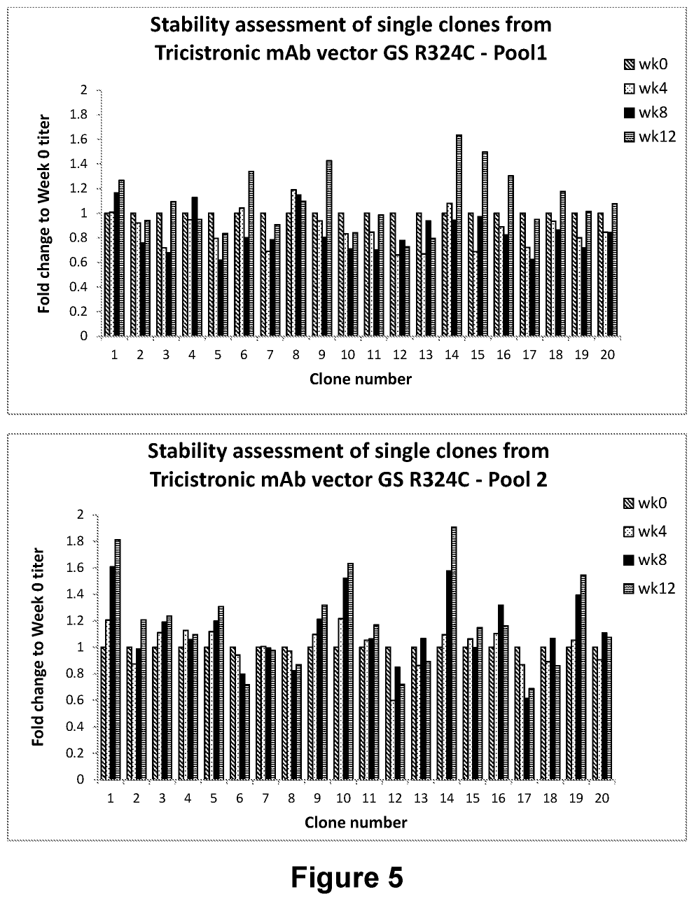

The methods to prepare GS constructs, measure GS activity, and measure luciferase activity were described in the Materials and Methods section. FIG. 4 represented an actual percentage of the GS activity normalized to that of WT GS activity. The GS activities, before luciferase normalization, were blanked with an untransfected CHO-K1 GS^{-/-} cell lysate. The expression variations across the GS constructs were normalized accordingly via their luciferase activities. It was noted that the GS activity assay might not be sensitive enough for very low GS activity such that the known R342C and R341C mutants have near blank level activities. Nevertheless, the attenuation caused by the different mutations can be clearly observed. This result was a representative of two independent experiments.

Stability Assessment of the mAb Producing Clones Selected Using the GS Mutant.

Stability assessment of 20 single clones derived from 2 stable pools transfected with Tricistronic mAb GS construct. The mAb production of these single clones was measured over 12 weeks (about 60 generations) to assess the stability of these clones. The mAb titer of week 12 was divided over that of week 0 to obtain the fold change. Typically, stability level of 70% and above is defined as an acceptable level of stability for the stable clone. The graph showed that almost all the single clones except for clone 17 of stable pool2 (68%) was above the 70% benchmark. This is a relatively high percentage (>95%) of clones with the desired level of stability. This results support the advantage of using attenuated GS as the selection marker as the selection pressure is constantly present. The single clones are isolated from the pools containing the R324C mutation.

Comparison of the mAb Titers Generated Using the Wildtype (wt) GS and GS Mutants as the Selection Markers.

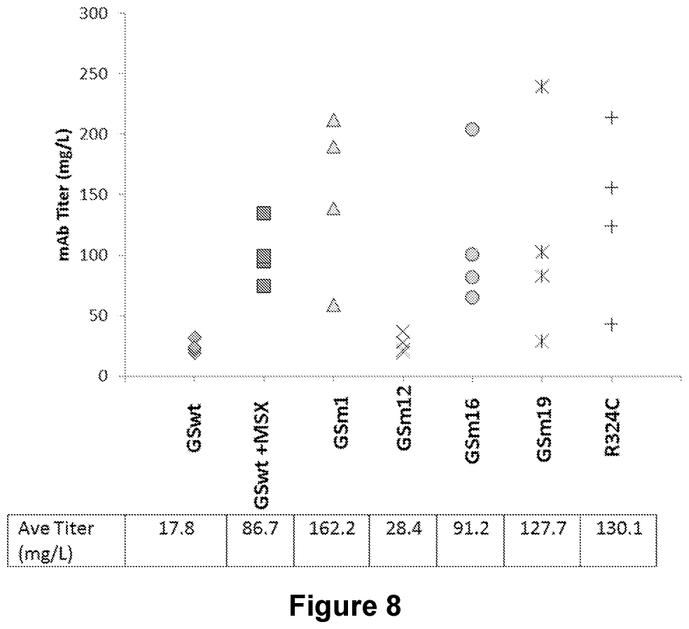

The selection marker was incorporated into the tricistronic vector expressing Antibody A and transfected into CHO-K1 GS^{-/-} cells. The selection was performed by removing L-glutamine from the media. For GSwt+MSX, after recovery from L-glutamine free media, the stable pools were then subjected to 25 μM MSX selection. The batch-culture titer for each stable pools generated is represented as a data point in the graph as shown on FIG. 8.

Stability Assessment of 5 Random Clones from Stable Pools Generated Using GSwt or GSm16 as the Selection Marker.

Stable pools of GA101 expressing CHO-K1 GS^{-/-} cells were generated using either the GSwt (wild type GS) or GSm16 (GS having S257A mutation site) selection marker in the tricistronic vector. Five random clones from each pool were scaled up for stability assessment in growth media without MSX and L-glutamine. Batch-culture titers were measure every 4 weeks. As depicted on FIGS. 9A-9B, the results showed that only 2 out of 5 GSwt clones were stable (i.e. maintained at least 70% of its original titer level) whereas 4 out of 5 GSm16 clones were stable.

1. An expression vector comprising a polynucleotide encoding an attenuated glutamine synthetase comprising a single amino acid substitution with alanine at position 63, 66, 134, 136, 162, 192, 196, 203, 248, 249, 253, 255, 257, 262, 299, 305, 319, 324, 333, 336, 338, 340, 341 of a wild type glutamine synthetase comprising the amino acid sequence set forth in SEQ ID NO: 1, 3, or 4.

2. The expression vector of claim 1, wherein the expression vector further comprises at least one polynucleotide encoding for a polypeptide of interest.

3. (canceled)

4. (canceled)

5. (canceled)

6. (canceled)

7. (canceled)

8. (canceled)

9. The expression vector of claim 1, wherein the polynucleotide encoding the attenuated glutamine synthetase is operatively linked to a promoter.

10. The expression vector of claim 2, wherein only one polynucleotide encoding the polypeptide of interest is operatively linked to a promoter.

11. The expression vector of claim 10, wherein the polynucleotide encoding the attenuated glutamine synthetase is not linked to a promoter.

12. The expression vector of claim 2, wherein the polypeptide of interest is selected from the group consisting of a recombinant protein and a part thereof, a fusion protein and a part thereof, an antibody and a part thereof.

13. The expression vector of claim 2, wherein the polypeptide of interest is the heavy chain of an antibody of interest and/or light chain of an antibody of interest.

14. The expression vector of claim 2, wherein the expression vector comprises two polynucleotides each encoding for a polypeptide of interest, wherein one of the polynucleotides encodes for the light chain of an antibody of interest, and one of the polynucleotides encodes for the heavy chain of the same antibody of interest.

15. The expression vector of claim 2, wherein the polynucleotide encoding for the light chain of an antibody of interest and the polynucleotide encoding for the heavy chain of the same antibody of interest are separated by polynucleotides encoding an internal ribosome entry site (IRES); and/or wherein the polynucleotide encoding for the heavy chain of an antibody of interest is separated from the polynucleotide encoding for a glutamine synthetase with reduced activity by polynucleotide encoding an internal ribosome entry site (IRES).

16. (canceled)

17. The expression vector of claim 15, wherein the polynucleotide encoding the internal ribosome entry site separating the polynucleotide encoding for the light chain of an antibody of interest and the polynucleotide encoding for the heavy chain of the same antibody of interest is polynucleotide encoding a wild type internal ribosome entry site (IRES), and wherein the internal ribosome entry site separating the polynucleotide encoding for the heavy chain of an antibody of interest from the polynucleotide encoding for a glutamine synthetase with reduced activity is polynucleotide encoding an internal ribosome entry site with attenuated translation efficiency (IRESatt).

18. (canceled)

19. (canceled)

20. A method of producing polypeptide of interest, comprising:
(a) transforming a host cell having no glutamine synthetase activity with the expression vector of claim 2;
(b) culturing the transformed host cell in a medium that selectively allows the production of polypeptide of interest; and
(c) collecting the polypeptide of interest from the medium of (b).

21. The method of claim 20, wherein the medium is a glutamine free cell culture medium.

22. The method of claim 21, wherein the method does not comprise the use of a glutamine synthetase inhibitor, optionally wherein the glutamine synthetase inhibitor is methionine sulphoximine (MSX).

23. A kit comprising the expression vector of claim 1.

24. The kit of claim 23, wherein the kit further comprises at least one of the following:
(i) a host cell having no glutamine synthetase activity; and
(ii) a transfection medium or means to carry out a transfection;
and/or wherein the kit further comprises a glutamine free cell culture medium.

25. (canceled)

26. An expression vector comprising a polynucleotide encoding an attenuated glutamine synthetase comprising a single amino acid substitution with alanine at position 305 of a wild type glutamine synthetase comprising the amino acid sequence set forth in SEQ ID NO: 1, 3, or 4.
